(19) United States
(12) Patent Application Publication
(10) Pub. No.: US 2004/0142381 A1
(43) Pub. Date: Jul. 22, 2004

(54) Methods for designing IGF1 receptor modulators for therapeutics

(76) Inventors: Stevan R. Hubbard, New York, NY (US); W. Todd Miller, East Setauket, NY (US); Jeffrey H. Till, Daly City, CA (US); Svetlana Favelyukis, La Jolla, CA (US)

Correspondence Address: KLAUBER & JACKSON, 411 HACKENSACK AVENUE, HACKENSACK, NJ, 07601

(21) Appl. No.: 10/632,139

(22) Filed: Jul. 31, 2003

Related U.S. Application Data
(60) Provisional application No. 60/400,001, filed on Jul. 31, 2002.

(51) Int. Cl. G01N 33/53; G06F 19/00; G01N 33/48; G01N 33/50

(52) U.S. Cl. 435/7.1; 702/19

(57) ABSTRACT

The present invention is directed to methods for designing ligands capable of binding to the kinase domain of the insulin-like growth hormone-1 (IGF-1) receptor with high affinity. Upon binding, these ligands are capable of acting as modulators of IGF1 activity. IGF1 modulators designed or identified using the methods of the invention may serve to inhibit an IGF1 activity or may serve to increase or prolong an IGF1 activity. Further provided are screening methods for identifying small molecules capable of binding to the tyrosine kinase domain of the IGF-I receptor.

### STATEMENT OF RELATED PATENT APPLICATION

This application claims priority under 35 USC § 119(e) to U.S. provisional application 60/400,001 filed 31 Jul. 2002, which application and the entire disclosure thereof is herein specifically incorporated by reference in its entirety.

### STATEMENT OF FEDERALLY SPONSORED RESEARCH

This invention was made in part with NIH/NIDDK Grant No. R01 DK52916 and NIH/NCI Grant No. PO1 CAO28146. The Federal Government may have certain rights in the invention.

### BACKGROUND OF THE INVENTION

1. Field of the Invention

The present invention pertains to the fields of molecular biology, and more particularly, to receptor signaling.

2. Statement of Related Art

The IGF1 receptor is an α₂β₂ transmembrane tyrosine kinase that is widely expressed in many human tissues and cell types. Binding of the secreted growth factor ligands IGF1 or IGF2 results in activation of the IGF1 receptor. IGF2, however, binds to the receptor with lower affinity relative to that of IGF1. Ligand binding to the α subunits in the extracellular domain induces changes in receptor conformation and triggers autophosphorylation of the cytoplasmic β subunits on specific tyrosine residues, alterations which stimulate catalytic activity and expose and/or create binding sites for downstream signaling proteins.

Under normal physiological conditions, the IGF1 receptor plays an important role in the regulation of cell growth and differentiation, and in protection from apoptosis. Disruption of the IGF1 receptor in mice leads to fetal growth retardation and abnormalities in the development of muscle, skin, bone, and the central nervous system. Elevated levels of the IGF1 receptor are observed in a variety of human tumor types, and interference with IGF1 receptor function—by antisense strategies, antibodies, or dominant-negative mutants—reverses the transformed phenotype in a variety of tumor cell lines. For these reasons, the IGF1 receptor has emerged as a therapeutic target for the treatment of human cancer.

Several publications and patent documents are referenced in this application in order to more fully describe the state of the art to which this invention pertains. The disclosure of each of these publications and documents is incorporated by reference herein.

### SUMMARY OF THE INVENTION

The present invention relates to methods for designing ligands capable of binding to the tyrosine kinase domain of the insulin-like growth factor-1 (IGF1) receptor to modulate catalytic activity and/or downstream signaling. Further, the invention relates to screening methods for identifying small molecules capable of binding to the tyrosine kinase domain of the IGF1 receptor. Modulators identified using the methods of the invention are capable of inhibiting or enhancing the catalytic activity and/or downstream signaling of the tyrosine kinase domain of the IGF1 receptor.

Accordingly, the present invention is directed to methods for designing ligands capable of binding to the tyrosine kinase domain of the IGF1 receptor to disrupt catalytic activity and/or downstream signaling. The invention also relates to screening methods for identifying small molecules capable of binding to and disrupting the tyrosine kinase domain of the IGF1 receptor.

The present invention also pertains to methods for designing ligands capable of binding to the tyrosine kinase domain of the IGF1 receptor to enhance catalytic activity and/or downstream signaling. Moreover, the invention also encompasses screening methods for identifying small molecules capable of binding to and activating and/or perpetuating the activity of the tyrosine kinase domain of the IGF1 receptor.

The present invention provides three-dimensional structural information relating to the IGF1 receptor which may be used advantageously in methods for designing small molecule modulators (i.e., inhibitors or activators) specific for the IGF1 receptor kinase (IGF1RK) domain. In addition, the invention provides the crystallographic phasing information necessary to determine crystal structures of the kinase domain in association with modulatory molecules.

In one aspect of the invention, computer-assisted methods are provided for selecting a compound capable of binding to the tyrosine kinase (TK) domain of the IGF1 receptor, comprising screening a plurality of compounds to determine the ability of a test compound to fit into a three-dimensional structure formed by the TK domain of the IGF1 receptor, and selecting a test compound from the plurality which is predicted to fit into the three-dimensional space.

In one embodiment of the invention, the three-dimensional structure is the TK domain of the IGF1 receptor described by the coordinates of APPENDIX A. In another embodiment, the tyrosine kinase domain of IGF1 comprises residues 956-1,256 of the human IGF1 receptor (residues 992-1292 of the sequence shown in SEQ ID NO: 1 which contains a leader sequence). In another embodiment, the computer-assisted methods are, for example, virtual ligand docking and screening techniques capable of designing and/or identifying a compound predicted to bind to a three-dimensional motif of the tyrosine kinase domain of the IGF1 receptor. A three-dimensional motif of the tyrosine kinase domain of the IGF1 receptor may comprise the ATP-binding pocket (comprising amino acid residues 975-984, 1001-1003, 1033-1034, 1049-1056, 1010-1012, 1122-1123; see Appendix A), or other regions/motifs such as, but not limited to, the peptide substrate binding groove in the C-terminal kinase lobe (comprising amino acid residues 1137-1157, 1105-1109; see Appendix A), the hinge region on the backside of the kinase domain (comprising amino acid residues 1025-1035, 1112-1121, 1050-1055; see Appendix A), and the alpha helix C (comprising amino acid residues 1005-1029; see Appendix A). Compounds may be designed and/or identified that are predicted to bind to three-dimensional motifs with a range of different affinities. In one embodiment, a compound is designed and/or identified that is predicted to bind to a three-dimensional motif with high affinity.

In an aspect of the invention, binding of a compound to a three-dimensional motif of the tyrosine kinase domain of the IGF1 receptor is predicted to modulate an activity of the IGF1 receptor. In one embodiment, modulating an activity of the IGF1 receptor reduces or inhibits an activity of the IGF1 receptor. Alternatively, modulating an activity of the IGF1 receptor increases or prolongs an activity of the IGF1 receptor. In one aspect of the invention, the activity which is modulated is the tyrosine kinase activity of the IGF1 receptor receptor

In a specific embodiment of the method of the invention, ligands capable of discriminating between the IGF1 receptor and the highly homologous insulin receptor are designed and/or identified by the computer assisted methods described below.

Compounds identified by the method of the invention are subsequently tested in in vitro assays as described below to determine their ability to modulate (i.e., inhibit or activate) an activity (e.g., tyrosine kinase activity) of the IGF1 and insulin receptors.

In a second aspect, the invention provides a computer-assisted method for designing a compound capable of binding to the TK domain of the IGF1 receptor, comprising determining the ability of a test compound to fit into a three-dimensional structure formed by the TK domain of the IGF1 receptor, and selecting a test compound predicted to bind the TK domain of the IGF1 receptor. In one embodiment, the three-dimensional structure is the tyrosine kinase domain of the IGF1 receptor described by the coordinates of APPENDIX A. In another embodiment, the tyrosine kinase domain of the IGF1 receptor comprises amino acid residues 992-1292 of SEQ ID NO: 1.

The computer-assisted method used may involve virtual ligand docking and screening techniques that are capable of designing and/or identifying a compound predicted to bind to a three-dimensional motif of the tyrosine kinase domain of the IGF1 receptor. A three-dimensional motif of the tyrosine kinase domain of the IGF1 receptor includes, but is not limited to, the ATP-binding pocket (comprising amino acid residues 975-984, 1001-1003, 1033-1034, 1049-1056, 1010-1012, 1122-1123; see Appendix A), the peptide substrate binding groove in the C-terminal kinase lobe (comprising amino acid residues 1137-1157, 1105-1109; see Appendix A), the hinge region on the backside of the kinase domain (comprising amino acid residues 1025-1035, 1112-1121, 1050-1055; see Appendix A), and the alpha helix C (comprising amino acid residues 1005-1029; see Appendix A). Compounds may be designed and/or identified that are predicted to bind to these three-dimensional motifs with a range of different affinities. In one embodiment, a compound is designed and/or identified that is predicted to bind to a three-dimensional motif with high affinity.

In an embodiment, binding of the test compound to the IGF1RK is predicted to modulate an IGF1 receptor activity. Modulation of an IGF1 receptor activity may be predicted to involve reducing and/or inhibiting an IGF1 receptor activity, or enhancing and/or prolonging an IGF1 receptor activity. The receptor activity to be modulated may comprise the tyrosine kinase activity of the IGF1 receptor.

In a third aspect, the invention provides a computer-assisted method for designing a molecule capable of modulating an activity of an IGF1 receptor, comprising determining the ability of a test molecule to fit into a three-dimensional structure formed by the TK domain of the IGF1RK, selecting the test molecule predicted to bind to IGF1RK, generating the test molecule, contacting the test molecule with the three-dimensional IGF1RK site, and determining if the test compound binds the IGF1RK. In one embodiment, the three-dimensional structure is the tyrosine kinase domain of the IGF1 receptor having coordinates of APPENDIX A. In another embodiment, the tyrosine kinase domain of the IGF1 receptor comprises amino acid residues 992-1292 of SEQ ID NO: 1.

In an embodiment, the computer-assisted method is virtual ligand docking and screening techniques capable designing and/or identifying a compound predicted to bind to a three-dimensional motif of the tyrosine kinase domain of the IGF1 receptor. A three-dimensional motif of the tyrosine kinase domain of the IGF1 receptor may comprise, without limitation, the ATP-binding pocket (comprising amino acid residues 975-984, 1001-1003, 1033-1034, 1049-1056, 1010-1012, 1122-1123; see Appendix A), the peptide substrate binding groove in the C-terminal kinase lobe (comprising amino acid residues 1137-1157, 1105-1109; see Appendix A), the hinge region on the backside of the kinase domain (comprising amino acid residues 1025-1035, 1112-1121, 1050-1055; see Appendix A), and the alpha helix C (comprising amino acid residues 1005-1029; see Appendix A). Compounds may be designed and/or identified that are predicted to bind to these three-dimensional motifs with a range of different affinities. In one embodiment, a compound is designed and/or identified that is predicted to bind to a three-dimensional motif with high affinity. A small molecule capable of binding to an IGF1RK and, for example, interfering with a function of a domain or region of the receptor important for receptor activity such as those indicated above is expected to act as an inhibitor of IGF1RK activity.

Alternatively, a small molecule capable of binding to an IGF1RK and, for example, promoting and/or activating a function of a three-dimensional motif (e.g., a domain or region) of the receptor important for receptor activity is expected to act as an activator of IGF1RK activity. Such domains or regions include, without limitation, regions which contribute to the formation and/or stability of an active three dimensional conformation of the tyrosine kinase domain (a catalytically competent form) and regions which are implicated in binding of downstream signaling molecules, particularly those signaling molecules that are involved in downregulating IGF1RK activity. Compounds identified that are capable of binding to a region(s) that contribute to the formation and/or maintenance of an active three-dimensional conformation may effectively stabilize the active receptor structure and thereby prolong receptor activity. Alternatively, compounds that interfere with the binding of downstream effector molecules whose association with the IGF1R leads to a decrease in kinase activity or a reduction in IGF1R signaling may also enhance IGF1R kinase activity. Compounds that stabilize the active receptor conformation and/or interfere with the binding of signaling molecules that downregulate IGF1R function, for example, are expected to promote IGF1 receptor kinase activity.

In one embodiment, a test compound is a non-peptide-based molecule. In another embodiment, a test compound is a peptide-based molecule.

Other features and advantages of the invention will be apparent from the detailed description, the drawings, and the claims.

### BRIEF DESCRIPTION OF THE DRAWINGS

FIG. 1 is a molecular surface representation of the TK domain of the IGF1RK illustrating surface differences between IGF1RK and IRK. The molecular surface contributed by side chains that differ between the two receptor structures is colored green. The molecular surface contributed by the Thr1053 side chain in the interlobe linker is colored yellow. Stick representations of the nucleotide analog (AMP-PCP) and peptide are shown.

FIG. 2 is a backbone worm representation of IGF1RK. Segments corresponding to residues that differ between IGF1RK and IRK are colored green. Stick representations of the nucleotide analog (AMP-PCP) and peptide are shown. The semi-transparent segment represents the portion of the kinase insert disordered in the structure.

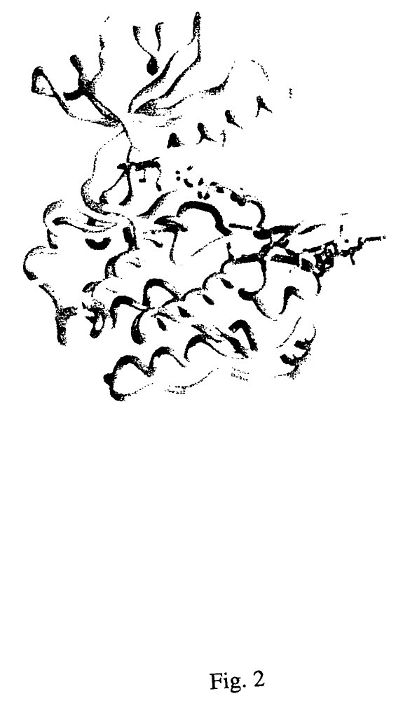

FIG. 3 is a ribbon diagram of the IGF1RK structure. P-strands (numbered) are shown in cyan; β-helices (lettered) are shown in red. The peptide is colored orange with the phosphate-acceptor Tyr shown in ball-and-stick representation. The nucleotide analog, AMP-PCP, is also shown in ball-and-stick representation (black). The dashed gray coil represents the disordered portion of the kinase insert. The N-terminal (NT) end of the structure is labeled. The C-terminal end is after αJ, hidden behind β8.

FIG. 4 shows the interactions within the A-loop in stereo. The A-loop (residues 1,123-1,145) is shown as a backbone worm (green) with side chains of selected residues shown in stick representation (carbon=green, nitrogen=blue, oxygen=red, sulfur=yellow, and phosphorus=black). Residues contributing to stabilization of the activation loop via hydrophobic interactions are shown with a molecular surface. Hydrogen bonds are shown as dashed lines (black).

FIG. 5 shows the interactions between the A-loop and other kinase segments in stereo. A backbone worn representation is shown for the A-loop (green), a segment including part of the catalytic loop (residues 1,100-1,105; in orange) and a segment corresponding to β12 (residues 1,157-1,159; in gray). Side chain and main chain atoms are shown in stick representation with the same color scheme as in FIG. 4 with the exception of carbon, which is colored the same as the corresponding backbone worm. For clarity, pTyr 1136 is omitted.

FIG. 6 depicts stereo views of interactions at the IGF1RK-peptide substrate interface. A semitransparent molecular surface (gray) of IGF1RK is shown with residues (labeled and displayed in stick representation) that define the peptide binding cleft and interact with the peptide substrate. The peptide (shown in stick representation) is illustrated without a molecular surface, with residues labeled relative to the phosphate acceptor Tyr (P0). Hydrogen bonds between the peptide and the enzyme are shown as black lines. Bond coloring is carbon=orange, oxygen=red, nitrogen=blue, sulfur=green, and phosphorus=yellow.

### DETAILED DESCRIPTION OF THE INVENTION

Before the present assay methodology and treatment methodology are described, it is to be understood that this invention is not limited to particular assay methods, or test compounds and experimental conditions described, as such methods and compounds may vary. It is also to be understood that the terminology used herein is for the purpose of describing particular embodiments only, and is not intended to be limiting, since the scope of the present invention will be limited only the appended claims.

Definitions

As used in this specification and the appended claims, the singular forms “a”, “an” and “the” include plural references unless the context clearly dictates otherwise. Thus for example, references to “the method” include one or more methods, and/or steps of the type described herein and/or which will become apparent to those persons skilled in the art upon reading this disclosure and so forth.

The term “amino acid” within the scope of the present invention and as used in its broadest sense, is meant to include the naturally occurring L alpha amino acids or residues. The commonly used one- and three-letter abbreviations for naturally occurring amino acids are used herein (Lehninger, Biochemistry, 2d ed., pp. 71-92, (Worth Publishers: New York, 1975). The term includes D-amino acids as well as chemically-modified amino acids such as amino acid analogs, naturally occurring amino acids that are not usually incorporated into proteins such as norleucine, and chemically-synthesized compounds having properties known in the art to be characteristic of an amino acid. For example, analogs or mimetics of phenylalanine or proline, which allow the same conformational arrangement of the peptide compounds as natural Phe or Pro, are included within the definition of amino acid. Such analogs and mimetics are referred to herein as “functional equivalents” of an amino acid. Other examples of amino acids are listed by Roberts and Vellaccio, The Peptides: Analysis, Synthesis, Biology, Eds. Gross and Meiehofer, Vol. 5, p. 341 (Academic Press, Inc.: N.Y. 1983). The term “amino acid” also has further, more detailed measuring as the latter pertains to the description of the invention, which usage and more detailed meaning is set forth in Paragraph 0080, infra.

The term “conservative” amino acid substitution as used herein to refer to amino acid substitutions that substitute functionally-equivalent amino acids. Conservative amino acid changes result in silent changes in the amino acid sequence of the resulting peptide. For example, one or more amino acids of a similar polarity act as functional equivalents and result in a silent alteration within the amino acid sequence of the peptide. The largest categories of conservative amino acid substitutions include: hydrophobic, neutral hydrophilic, polar, acidic/negatively charged, neutral/charged, basic/positively charged, aromatic, and residues that influence chain orientation. One of ordinary skill in the art is aware of the amino acid residues that are categorized within any one of the above categories and may, therefore, be conservatively substituted. In addition, “structurally-similar” amino acids can substitute conservatively for some of the specific amino acids. Groups of structurally similar amino acids include: Leu, and Val; Phe and Tyr; Lys and Arg; Gln and Asn; Asp and Glu; and Gly and Ala. In this regard, it is understood that amino acids are substituted on the basis of side-chain bulk, charge, and/or hydrophobicity. Amino acid residues are classified into four major groups: acidic, basic, neutral/non-polar, and neutral/polar.

An acidic residue has a negative charge due to loss of an H ion at physiological pH and is attracted by aqueous solution so as to seek the surface positions in the conformation of a peptide in which it is contained when the peptide is in aqueous solution.

A basic residue has a positive charge due to association with an H ion at physiological pH and is attracted by aqueous solution so as to seek the surface positions in the conformation of a peptide in which it is contained when the peptide is in aqueous medium at physiological pH.

A neutral/non-polar residue is not charged at physiological pH and is repelled by aqueous solution so as to seek the inner positions in the conformation of a peptide in which it is contained when the peptide is in aqueous medium. These residues are also designated “hydrophobic residues.”

A neutral/polar residue is not charged at physiological pH, but the residue is attracted by aqueous solution so as to seek the outer positions in the conformation of a peptide in which it is contained when the peptide is in aqueous medium.

“Amino acid” residues can be further classified as cyclic or non-cyclic, and aromatic or non-aromatic with respect to their side-chain groups, these designations being commonplace to the skilled artisan.

Peptides of the invention can be synthesized by standard solid-phase synthesis techniques. Such peptides are not limited to amino acids encoded by genes for substitutions involving the amino acids. Commonly encountered amino acids that are not encoded by the genetic code include, for example, those described in WO 90/01940, as well as, for example, 2-amino adipic acid (Aad) for Glu and Asp; 2-aminopimelic acid (Apm) for Glu and Asp; 2-aminobutyric (Abu) acid for Met, Leu, and other aliphatic amino acids; 2-aminoheptanoic acid (Ahe) for Met, Leu, and other aliphatic amino acids; 2-aminoisobutyric acid (Aib) for Gly; cyclohexylalanine (Cha) for Val, Leu and Ile; homoarginine (Har) for Arg and Lys; 2,3-diaminopropionic acid (Dpr) for Lys, Arg, and His; N-ethylglycine (EtGly) for Gly, Pro, and Ala; N-ethylglycine (EtGly) for Gly, Pro, and Ala; N-ethylasparagine (EtAsn) for Asn, and Gin; hydroxylysine (Hyl) for Lys; allohydroxylysine (AHyl) for Lys; 3-(and 4-)hydroxyproline (3Hyp, 4Hyp) for Pro, Ser, and Thr; allo-isoleucine (Alle) for lie, Leu, and Val; .rho.-amidinophenylalanine for Ala; N-methylglycine (MeGly, sarcosine) for Gly, Pro, and Ala; N-methylisoleucine (Melle) for Ile; norvaline (Nva) for Met and other aliphatic amino acids; norleucine (Nle) for Met and other aliphatic amino acids; ornithine (Orn) for Lys, Arg and His; citruline (Cit) and methionine sulfoxide (MSO) for Thr, Asn, and Gin; and N-methylphenylalanine (MePhe), trimethylphenylalanine, halo-(F—, Cl—, Br—, or I) phenylalanine, or trifluorylphenylalanine for Phe.

The term “tyrosine kinase domain of the insulin-like growth factor 1”, “the IGF1RK site”, and/or “the IGF1RK domain” and the like, refer to a protein fragment having the three-dimensional structure as described in attached APPENDIX A. The IGF1RK site is also defined as comprising amino acid residues 956-1,256 of the human IGF1RK, corresponding to Trp (W) 992-1292 of the amino acid sequence of SEQ ID NO: 1, which comprises the amino acid sequence of the IGF1 receptor including a 36 amino acid leader sequence.

As used herein, the term “modulator” refers to a compound capable of modulating, altering, or changing an activity of a molecule. In the context of the present invention, a modulator may be used to alter an activity of the IGF1 receptor or a functional fragment thereof. In a particular embodiment, a modulator may alter an activity associated with the tyrosine kinase (TK) domain of the IGF1 receptor or a fragment of the receptor comprising the TK domain. The term “modulator”, “modulatory compound”, or “modulatory agent” encompasses a compound/agent capable of decreasing, inhibiting, and/or reducing an activity of a molecule (i.e., an inhibitor) or increasing, enhancing, and/or prolonging an activity of a molecule (i.e., an activator).

An inhibitor of the TK domain of the IGF1 receptor, for example, is a compound/agent capable of decreasing, inhibiting, and/or reducing an activity of the TK domain of the IGF1 receptor. It is to be understood that a compound/agent capable of inhibiting the TK domain of the IGF1 receptor may be specific for an activity of the IGF1 TK domain or may be able to act as an inhibitor of TK domains derived from other kinases.

An activator of the TK domain of the IGF1 receptor, for example, is a compound/agent capable of increasing, enhancing, and/or prolonging an activity of the TK domain of the IGF1 receptor. It is to be understood that a compound/agent capable of “activating” or “prolonging the activated state” of the TK domain of the IGF1 receptor may be specific for an activity of the IGF1 TK domain or may be able to act as an activator of TK domains derived from other kinases.

As used herein, a “three-dimensional motif” refers to a spatial conformation formed by an association or arrangement of different amino acid residues and/or regions of a molecule. The nature of such associations and arrangements is discussed in detail in Examples 1-4 herein below.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated by reference to disclose and describe the methods and/or materials in connection with which the publications are cited.

General Description

The IGF system includes membrane-bound receptors for IGF-1, IGF-2, and insulin. The Type 1 IGF (IGF1) receptor is closely related to the insulin receptor in structure and shares some of its signaling pathways (Jones and Clemmons (1995) Endocr. Rev., 16: 3-34). The IGF-2 receptor is a clearance receptor that does not appear to transmit an intracellular signal (Jones and Clemmons, supra).

As described herein, the IGF1 receptor is a key factor in normal cell growth and development (Daughaday and Rotwein (1989) Endocrine Rev. 10:68-91). Increasing evidence suggests, however, that IGF1 receptor signaling also plays a critical role in tumor cell growth, cellular transformation, and tumorigenesis (Baserga (1995) Cancer Res. 55:249-252; for a review, see Khandwala et al. (2000) Endocr. Rev. 21: 215-244). Key examples include loss of metastatic phenotype of murine carcinoma cells by treatment with antisense RNA to the IGF1 receptor (Long et al. (1995) Cancer Res., 55:1006-1009) and the in vitro inhibition of human melanoma cell motility (Stracke et al. (1989) J. Biol. Chem. 264:21554-21559) and of human breast cancer cell growth by the addition of IGF1 receptor antibodies (Rohlik et al. (1987) Biochem. Biophys. Res. Commun. 149:276-281).

Moreover, the IGFs have been shown to be potent breast cancer cell mitogens. This finding is based, in part, on the observation that IGF-1 enhances breast cancer cell proliferation in vitro (Cullen et al. (1990) Cancer Res. 50:48-53). Since many breast cancers express both an IGF and IGF1 receptor, these cells possess all the required effectors for an autocrine proliferative loop (Quinn et al. (1996) J. Biol. Chem. 271:11477-11483; Stelleret al. (1996) Cancer Res., 56:1761-1765). Because breast cancer is a common malignancy affecting approximately one in every eight women and is a leading cause of cancer-related death in North American women (LeRoith et al. (1995) Ann. Int. Med., 122:54-59), new rational therapies are required for intervention. Since IGF1 can suppress apoptosis, cells lacking IGF1 receptors or having compromised IGF1 receptor signaling pathways may give rise to tumor cells that selectively die via apoptosis (Long et al. (1995) Cancer Res 55:1006-1009). Furthermore, it has become evident that alterations in IGF signaling in the context of other disease states, such as diabetes, may be responsible for exacerbating the complications of retinopathy (Smith et al. (1997) Science 276:1706-1709) and nephropathy (Homey et al. (1998) Am. J. Physiol. 274: F1045-F1053).

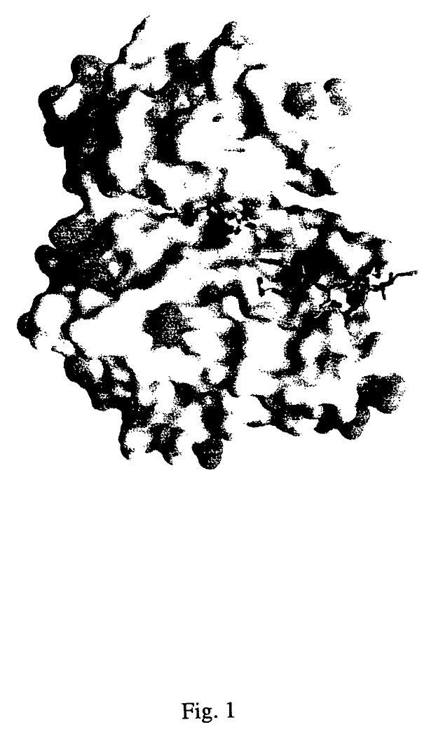

The present invention is based in part on the determination of the three-dimensional structure of the activated form of the tyrosine kinase domain of the IGF1 receptor, as determined by x-ray crystallography. The invention, therefore, provides a structural basis for understanding substrate recognition by the IGF1 receptor kinase domain (IGF1RK) and for designing small molecule modulators (i.e., inhibitors or activators) of IGF1RK. As described, the form of IGF1RK crystallized herein is the tris(3)-phosphorylated, activated form, and the structure contains a bound ATP analog and 14-residue peptide substrate (FIG. 1). The present inventors published the novel structure of the activated form of the TK domain of the IGF1 receptor (Favelyukis et al. (2001) Nature Structural Biology 8: 1058-1063), which publication is herein specifically incorporated by reference in its entirety.

A related crystal structure of IGF1RK has also been reported by Pautsch et al. (2001) Structure 9:955-965, which publication is herein specifically incorporated by reference in its entirety. The Pautsch et al. structure is of a partially activated, bis(2)-phosphorylated form having a bound ATP analog but no substrate peptide. A comparison of the crystal structures of the tris- and bis-phosphorylated forms of the IGF1 TK domain reveals that significant differences exist between these forms. An understanding of these differences can be applied to methods for screening/identifying small molecule modulators (i.e., inhibitors or activators) capable of interacting with the different structural forms of the TK domain of the IGF1 receptor.

The three-dimensional structure of the IGF1RK has also been determined in the unphosphorylated, low activity state by Munshi et al. (2002) J Biol Chem 277:38797-38802, which publication is herein specifically incorporated by reference in its entirety. A comparision of the crystal structures of the unphosphorylated, tris-, and bis-phosphorylated forms of the IGF1 TK domain may also be used to advantage to design/identify small molecule modulators of the IGF1R.

Of note, the IGF1 receptor is structurally homologous to the insulin receptor. The members of this receptor subfamily are hetero-tetrameric glycoproteins consisting of two extracellular ligand-binding α-subunits and two transmembrane catalytic β-subunits. The cytoplasmic portions of the 1-subunits possess a juxtamembrane region, a tyrosine kinase domain, and a C-terminal tail (Hubbard & Till (2000) Ann. Rev. Biochem. 69:373-398). The tyrosine kinase domains of the IGF1 and insulin receptors possess 84% sequence identity, while the juxtamembrane and C-terminal regions share 61% and 44% sequence identity, respectively (Blakesley et al. (1996) Cytokine Growth Factor Rev. 7: 153-159; Ullrich et al. (1986) EMBO J. 5:2503-2512). Despite this high degree of homology, the two receptors have distinct biological roles. While the insulin receptor is known to be a key regulator of physiological processes such as glucose transport and biosynthesis of glycogen and fat, the IGF1 receptor is a potent regulator of cell growth and differentiation (Blakesley et al. (1996) supra; Lammers et al. (1989) EMBO J. 8:1369-1375).

Ligand binding to the extracellular α-subunit of the IGF1 (or insulin) receptor triggers autophosphorylation of three tyrosine residues in the activation loop (A-loop) within the kinase domain of the P-subunit of the receptor (Kato et al. (1993) J. Biol. Chem. 268:2655-2661; Murakami et al. (1991) J. Biol. Chem. 266:22653-22660) resulting in an increase in catalytic activity (Wei et al. (1995) J. Biol. Chem. 270:8122-8130; Butler et al. (1998) Comp. Biochem. Physiol. 121:19-26). The three-dimensional structure of the insulin receptor tyrosine kinase domain (IRK) has been determined in the unphosphorylated, low activity state (Hubbard et al. (1994) Nature 372:746-754) and the tris-phosphorylated, high activity state (Hubbard et al. (1997) EMBO J. 16:5572-5581). These crystal structures show that autophosphorylation causes a major conformational change in the A-loop, resulting in unrestricted access of ATP and protein substrates to the kinase domain.

The molecular mechanism of IGF1 receptor activation has not been well characterized. The signaling differences between the insulin and IGF1 receptors may be due to structural/enzymatic differences within the tyrosine kinase domains. Alternatively, the differences in signaling may arise from other regions of the receptors that act in conjunction with the kinase domains.

As described in Examples 1-4, steady-state kinetic measurements were carried out on the differentially phosphorylated forms of the IGF1 receptor kinase domain (IGF1RK). As shown herein below, the crystal structure of a tris-phosphorylated IGF1RK in complex with a peptide substrate and an ATP analog was determined. These results provide a basis for comparison between the activation mechanisms of the two receptors.

In view of the above, the present discovery also presents evidence that may be applied to the identification of small molecule modulators (i.e., inhibitors or activators) that bind differentially to the TK domains of the insulin receptor and the IGF1 receptor. Inasmuch as different disorders/diseases are associated with altered responses/regulation of one of these receptors, there is a critical need to identify compounds capable of specifically modulating the kinase activity either the IGF1 or the insulin receptor. Such a compound would, therefore, not substantially affect the activity of the other related receptor tyrosine kinases.

A skilled artisan would also appreciate that the novel crystal structure of the activated IGF1R tyrosine kinase domain could be used to advantage to design of and/or screen for small molecule modulators of other related tyrosine kinases. Indeed, the structural information pertaining to the activated IGF1R TK domain could readily be extrapolated to predict the crystal structures of activated TK domains of related tyrosine kinases. Such in silico structural determinations may then be applied to the design of and/or screening for molecules that bind and potentially modulate an activity of a kinase domain(s).

Autophosphorylation of IGF1 Receptor Kinase

To characterize the autoregulatory features of IGF1RK, kinetic and structural analyses were carried out as described in Examples 1-4 below. IGF1RK (residues 956-1,256) was produced in Sf9 cells using a baculovirus expression vector. This portion of the IGF1 receptor is homologous to the region of the insulin receptor kinase crystallized (IRK); it contains the kinase domain and excludes the first 27 residues (the juxtamembrane region) and the last 81 residues (the C-terminal tail) of the cytoplasmic domain. IGF1RK was purified from insect cells in its unphosphorylated, low activity state. Upon addition of ATP, IGF1RK is activated by three discrete phosphorylation events, which result in the appearance of three differentially phosphorylated forms that can be resolved by non-denaturing gel electrophoresis.

To determine whether autophosphorylation of IGF1RK was intra- or intermolecular, a continuous spectrophotometric assay was used that couples production of ADP to oxidation of NADH, measured as a decrease in absorbance at 340 nm. Varying concentrations of the unphosphorylated form (0P) of IGF1RK were incubated with ATP. The time course of IGF1RK autophosphorylation shows a lag in ATP turnover that corresponds to the induction time for IGF1RK autophosphorylation. A time course analysis of induction times revealed that the phosphorylation reaction was concentration dependent. These results indicate that autophosphorylation of IGF1RK is an intermolecular event.

The mono-, bis-, and tris-phosphorylated forms of IGF1RK were purified to homogeneity using ion exchange chromatography. For the tris-phosphorylated (3P) form, the stoichiometry of phosphorylation was confirmed by MALDI-TOF mass spectrometry, which showed that all three sites of autophosphorylation are in the kinase A-loop. Based on peptide mapping experiments, the order of phosphorylation for IGF1RK is consistent with that determined for the insulin receptor. The first site of autophosphorylation is predominantly Tyr 1,135, followed by Tyr 1,131 and then by Tyr 1,136 (the corresponding residues in IRK are 1,158, 1,162, and 1,163).

The production of purified 0P, 1P, 2P, and 3P forms of IGF1RK, as described for the first time herein, facilitated a determination of the impact of each autophosphorylation event on the steady-state kinetic parameters of the insulin-like growth factor receptor tyrosine kinase. Kinetic experiments were performed using the peptide KKEEEEYMMMMG (SEQ ID NO: 2), a peptide identified by peptide library studies as being an optimal substrate for IRK (Songyang et al. (1995) Nature 373:536-539). These analyses, carried out using a continuous spectrophotometric assay, indicate that each phosphorylation event causes an increase in enzyme turnover number and a decrease in the Kₘ values for ATP and peptide substrate (Table 1). The overall increase in catalytic efficiency (Vₘₐₓ/Kₘ) of the 3P form, as compared to that of the 0P form, was >120-fold. The effects on Vₘₐₓ were most pronounced in the transition from the 0P to the 1P form of IGF1RK, while the largest effects on Kₘ occurred after the second autophosphorylation (Table 1). These results are consistent with a recent study comparing the kinetic parameters of the 0P and 3P forms of IRK.

For maximal IGF1RK catalytic activity, the distal ends of the A-loop must be properly positioned to bind MgATP and peptide substrate, respectively. By comparison to the unphosphorylated IRK structure (Hubbard et al. (1994) supra), in which the A-loop adopts an autoinhibitory conformation with Tyr 1,162 (Tyr 1,135 in IGF1RK) bound in the active site, phosphorylation of the A-loop tyrosine residues in IGF1RK incrementally destabilizes the autoinhibited form of the A-loop and stabilizes the catalytically competent form associated with the tris-phosphorylated IRK and IGF1RK structures. Based on the structural data for IRK and IGF1RK, destabilization of the autoinhibitory A-loop conformation derives from autophosphorylation of Tyr 1,135 and Tyr 1,131, and stabilization of the catalytically optimized A-loop conformation results from autophosphorylation of Tyr 1,136, and to a lesser extent, of Tyr 1,135. The effects on the steady-state kinetic parameters (Kₘ and Vₘₐₓ) of A-loop disordering and reordering upon stepwise autophosphorylation are complex. The significant decrease in the substrate Kₘ values in the transition from the 1P state (mainly Tyr 1,135) to the 2P state (Tyr 1,135 and Tyr 1,131) suggests that autophosphorylation of Tyr 1,135 is necessary but not sufficient to destabilize the autoinhibitory A-loop conformation; full destabilization requires autophosphorylation of Tyr 1,131 as well.

TABLE 1
KINETIC PROPERTIES OF IGF1RK

| Phosphorylation | ATP Kₘ | Peptide Kₘ | Vₘₐₓ | Vₘₐₓ/Kₘ |
|---|---|---|---|---|
| state | (μM) | (mM) | (μmol/min/mg) | peptide |
| 0P | 720 ± 39 | 1.3 ± 0.21 | 2.1 ± 1 | 1.6 |
| 1P | 527 ± 38 | 0.62 ± 0.07 | 12.7 ± 0.1 | 20 |
| 2P | 148 ± 11 | 0.13 ± 0.02 | 15.3 ± 0.5 | 118 |
| 3P | 107 ± 11 | 0.12 ± 0.01 | 23.6 ± 0.7 | 197 |

Structural Analysis of the Tris-Phosphorylated Form of IGF1RK

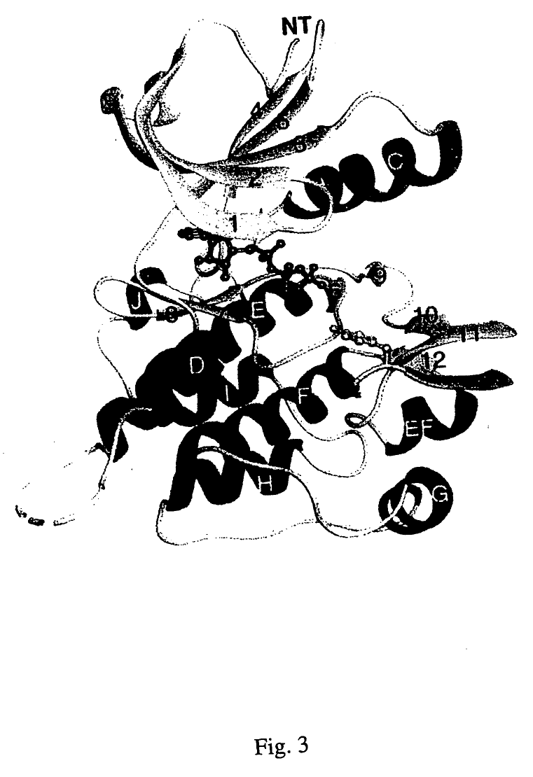

As described herein, the 2.1 Å-resolution crystal structure of the tris-phosphorylated, activated form of IGF1RK in complex with nucleotide analog AMP-PCP (β,γ-methyleneadenosine 5′-triphosphate) and peptide substrate was determined. IGF1RK shares the well-conserved architecture of a typical member of a tyrosine kinase family, consisting of two major subdomains, an amino-terminal (NT) lobe comprised of five anti-parallel β-strands (β1-β5) and a single α-helix (αC), and a carboxy-terminal (CT) lobe comprised of eight α-helices (αD-α.J) and three pairs of anti-parallel β strands (β7-β8, β6-β9, β10-β12). The nucleotide analog is situated in the cleft between the two lobes, and the peptide substrate is bound to the CT lobe, with the phosphate-acceptor Tyr hydrogen bonded to conserved residues in the catalytic loop (residues 1,103-1,110) (FIG. 3). The phosphorylated A-loop (residues 1,123-1.144) is well ordered and anchored to the CT lobe in a conformation that facilitates substrate binding and catalysis.

The overall structure of IGF1RK is similar to the structure of the activated, tris-phosphorylated form of the insulin receptor kinase (IRK). Superimposition of the Cα atoms in the CT lobe (IGF1RK residues 1,053-1,256) yields a root-mean-square deviation (rmsd) of only 0.6 Å. With residues in the CT lobe superimposed, a difference in the positions of the NT lobes of the two kinases is apparent. Relative to IRK, the NT and CT lobes of IGF1RK are approximately 7° more “open”. This difference that may result, in part, from crystal packing forces rather than intrinsic differences between the kinases. When the Cα atoms of the NT lobe of IRK and IGF1RK are superimposed, the rmsd is 1.2 Å, with the largest deviations found in αC, due to a relative displacement of αC vis-a-vis the β-sheet; in IRK, αC is slightly closer to the CT lobe.

A structural comparison of the 0P and 3P forms of IRK reveals a change in the relative lobe disposition as well as a reconfiguration of the A-loop. Upon A-loop phosphorylation and nucleotide binding, the NT lobe of IRK undergoes a rotation towards the CT lobe, resulting in closure of the catalytic cleft. In addition, αC undergoes an independent rotation (with respect to the β-sheet) towards the CT lobe. In the IGF1RK structure, αC is in approximately the same rotational position as in the tris-phosphorylated IRK structure, juxtaposing protein kinase-conserved Lys 1,003 (p3) and Glu 1,020 (αC).

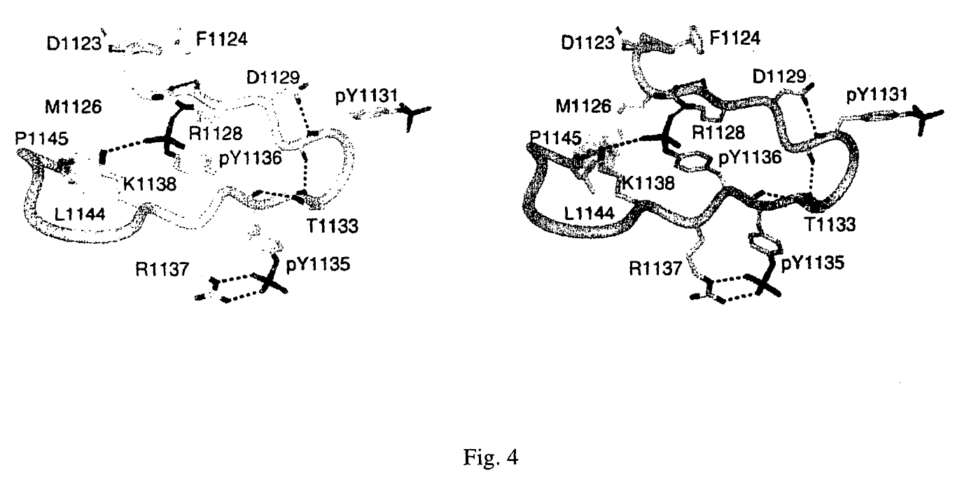

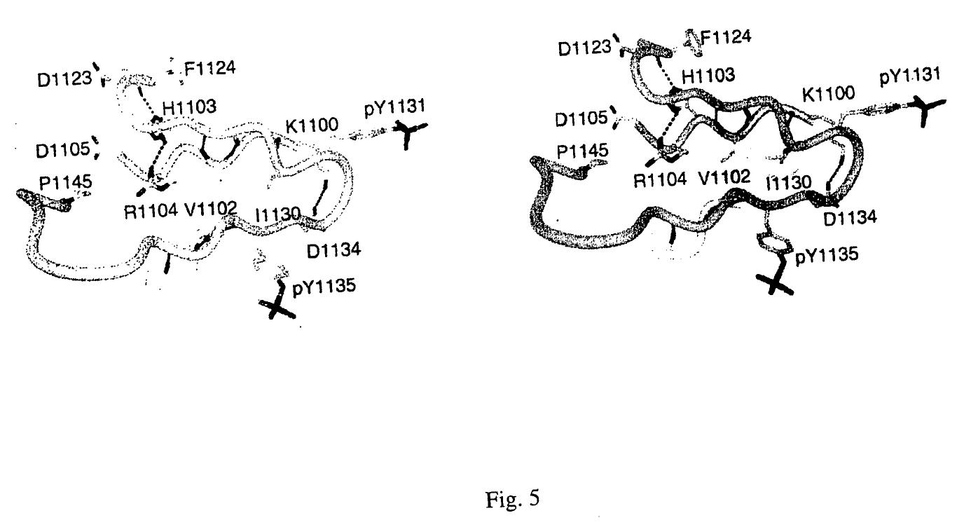

The conformation of the tris-phosphorylated A-loop in IGF1RK is stabilized by numerous interactions within the A-loop and between the A-loop and other segments of the kinase (FIGS. 4-5). The phosphate group of pTyr 1,135 is salt bridged to Arg 1,137, and the phosphate group of pTyr 1,136 is salt bridged to conserved Arg 1,128 and Lys 1,138 (FIG. 4). The pTyr 1,136-Arg 1,128 interaction is a key structural element in the pTyr-mediated stabilization of the A-loop. Interestingly, the pTyr 1,136-Lys 1,138 interaction was not observed in the IRK structure. Met 1,126, located immediately after the protein kinase-conserved DFG sequence at the beginning of the A-loop, packs against Leu 1,144 and tyrosine kinase-conserved Pro 1,145 at the end of the A-loop. Hydrophobicity is conserved in tyrosine kinases at positions 1,126 and 1,144 of the A-loop. Another conserved hydrophobic residue in the A-loop, Ile 1,130, packs against Val 1,102, the residue that precedes the catalytic loop (FIG. 5). Conserved His 1,103 and Arg 1,104 in the catalytic loop are hydrogen bonded to main-chain carbonyl oxygen atoms in the A-loop. Asp 1,134 in the A-loop is salt bridged to Lys 1,100 in the segment prior to the catalytic loop. In tyrosine kinases, there is a strong sequence correlation between Lys at position 1,100 and Asp or Glu at position 1,134. Additional stability for this A-loop configuration is derived from two pairs of short β-strand interactions (β9-β6 and β10-β12) (FIG. 5). As in the IRK structure, the first pTyr in the A-loop, pTyr 1,131, is exposed and makes no contacts with other residues.

Due to incomplete lobe closure, interactions between the nucleotide analog and protein are almost exclusively mediated through the adenine; the ribose hydroxyl group (O2′) is not within hydrogen-bonding distance of Asp 1,056 of the CT lobe, an interaction observed in the IRK structure. The B-factors of the nucleotide analog are high compared to those for protein atoms. The adenine moiety has the lowest B-factors of the nucleotide analog, making two hydrogen bonds to main-chain atoms in the interlobe linker and hydrophobic contacts with residues in the NT lobe of the cleft. The phosphate groups have the highest B-factors in the analog. The α-phosphate is hydrogen bonded to the main-chain nitrogen of Ser 979 (nucleotide-binding loop) and to the side chain of conserved Lys 1,003 via a water molecule. The β- and γ-phosphates appear to adopt more than one conformation. Although Mg²⁺ was included in the crystallization conditions, the electron density in the expected area of binding (to Asn 1,110 and Asp 1,123) is ambiguous, and therefore no Mg²⁺ ions are included in the final atomic model. The differences in ATP analog binding observed in the IGF1RK (AMP-PCP) and IRK (AMP-PNP) structures are probably due to differences in lobe closure, which can be influenced by crystal packing, rather than the particular analog used or intrinsic differences between the two kinases.

A synthetic peptide (KKKSPGEYVNIEFG) (SEQ ID NO: 3), which was based on the IRS-1 phosphorylation site Tyr 895, was co-crystallized with IGF1RK. This peptide was chosen for crystallization experiments because it had the lowest Km for IGF1R of a series of peptides derived from the IGF1R substrate IRS-1 (Xu et al., (1995) J. Biol. Chem. 270:29825-29830). Moreover, the structure of IRK was determined with a related peptide derived from IRS-1. The Km for this peptide was determined to be 128 μM for IGF1RK. Eight residues of the peptide are well defined by electron density (P−2 to P+5, where P0 is the phosphate-acceptor Tyr), and the overall mode of peptide binding to IGF1RK is very similar to that observed in the ternary IRK structure (Hubbard et al. (1997) supra). Val(P+1) to Phe(P+5) form a short anti-parallel α-strand which is paired with residues 1,140-1,144 of the A-loop (β11 in FIG. 3). The hydroxyloxygen of Tyr(P0) is hydrogen bonded to the side chains of two conserved residues in the catalytic loop, Asp 1,105 and Arg 1,109, and is in position for phosphoryl transfer. The side chains of the P+1, P+3 and P+5 residues of the peptide substrate fill a groove comprised primarily of hydrophobic amino acids in the CT lobe (FIGS. 8 and 9). The side chain of Phe(P+5) is in van der Waals contact with Gly 1,157 and packs against Ile(P+3), which in turn packs into a hydrophobic pocket comprised of Leu 1,154 (αEF), Met 1,149 (β11-αEF loop), and Leu 1,144 (β11). Interestingly, Gly 1,157 is conserved in the insulin receptor and Src, but in many tyrosine kinases the amino acid at this position is arginine (Arg). An Arg side chain would clearly alter the path of the peptide after the P+3 residue.

The side chain of Val(P+1) is too short to plug fully into the hydrophobic cavity formed by CT lobe residues Val 1,146 (β11-αEF loop), Leu 1,192 (αG) and Asn 1,188 (αG). In the ternary IRK structure, the peptide substrate contains a methionine (Met) at the P+1 and P+3 positions. The longer Met side chain at P+1 makes additional hydrophobic contacts relative to Val(P+1) in the IGFR1K structure. The IGF1RK structure suggests that a Met or a phenylalanine (Phe) would be optimal at the P+1 position, an observation supported by peptide substrate library studies for IRK (Songyang et al. (1995) supra). The residues involved in peptide substrate recognition are conserved between the insulin and IGF1 receptors. Thus, differences in substrate specificity are likely to arise from interactions outside the P-2 to P+5 region observed here. The relatively low Kₘ of the IRS-1 Tyr 895-derived peptide, with respect to other IRS-1 peptides tested (Xu et al. (1995) supra), may be due in part to the hydrophobic packing of Phe(P+5) between isoleucine [Ile(P+3)] and Gly 1,157.

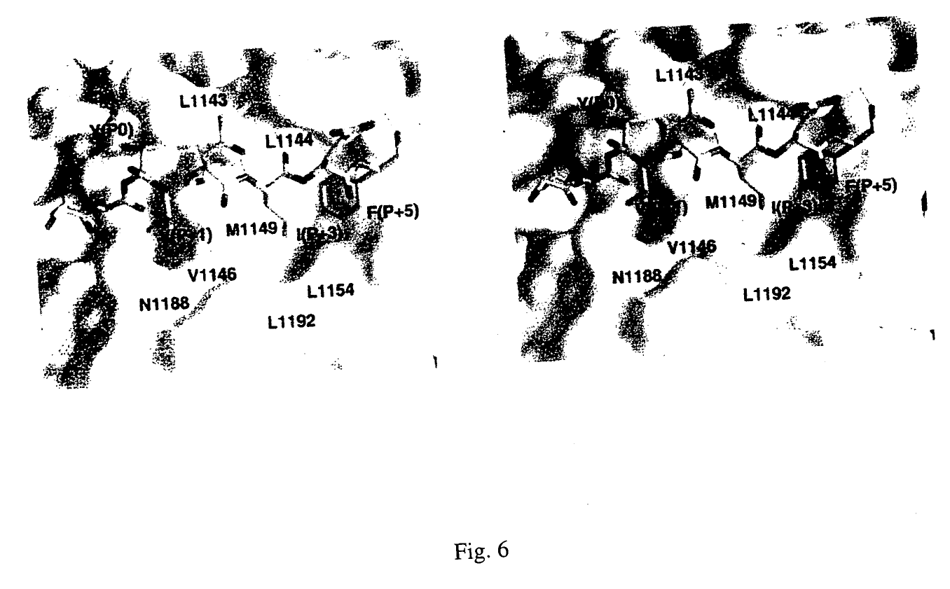

A majority of the kinase insert region of IGF1RK is disordered in the crystal structure (residues 1,069-1,076) and was not included in the atomic model. The sequences of the kinase inserts of IGF1RK and IRK are divergent. In the crystal structure of IRK, the kinase insert (residues 1,091-1,105) is ordered, stabilized in part by a salt bridge between Arg 1,101 in the insert and Glu 1,108 (αE). The corresponding residues in IGF1RK are Leu 1,074 and Lys 1,081. Although the functional roles in cell signaling for the kinase inserts in the insulin and IGF1 receptors have not been determined, these differences in sequence and structure may be significant. The kinase insert in IRK contains a proline (Pro) repeat conforming to the PXXP motif (residues 1,099-1,102) recognized by SH3 domains. In IGF1RK, the second Pro in this motif is missing. Moreover, a cluster of residues which differs between the two kinases is located near the kinase insert region (FIG. 6), which could provide specificity for protein-protein interactions.

Mapping the sequence differences between IGF1RK and IRK onto the IGF1RK structure highlights the conserved nature of the ATP-binding cleft, the A-loop, and the residues that directly interact with peptide substrate (FIG. 6). Residues that vary between the two kinases are found primarily on the kinase surface and do not alter the overall secondary or tertiary structure. Developing small molecule inhibitors that are specific for IGF1RK and do not affect IRK activity, therefore, poses a significant challenge. Typically, small molecule inhibitors of protein kinases have targeted the relatively well-conserved ATP-binding site, exploiting subtle sequence differences in this region to attain specificity (Mohammadi et al. (1997) Science 276:955-960 and (1998) EMBO J. 17:5896-5904; Schindler et al. (2000) Science 289:1938-1942; Zhu et a. (1999) Structure Fold. Des. 7:651-661). Within the ATP-binding cleft proper, the sequence identity between IGF1RK and IRK is 100%. As supported by evidence presented herein, the interlobe linker, however, is a good candidate target. The interlobe linker of IGF1RK comprises Thr 1,053 and Arg 1,054, which replace Ala 1,080 and His 1,081 of the IRK interlobe linker (FIG. 6). The distance from the methyl group of the Thr 1,053 side chain to the adenine of AMP-PCP is 6.7 Å, within reach of an ATP-competitive inhibitor.

Because the ATP binding pocket of protein kinases is generally well conserved, some attempts have been made to find or design inhibitors that target other regions of the kinase, particularly those involved in protein substrate recognition (Blum et al. (2000) Biochemistry 39:15705-15712; Parang et al. (2001) Nature Struct. Biol. 8:37-41). Since the structure of IGF1RK reveals that the residues involved in peptide binding in the immediate vicinity of the substrate tyrosine are identical to those in IRK, specificity for protein substrate phosphorylation must, therefore, be influenced by residues outside of this core region. Such residues would provide supplemental recognition information with which these kinases discriminate between their appropriate peptide substrates, thereby resulting in the differential substrate specificities observed for IGF1R and IRK. As indicated herein, the identification of such sites can be exploited in the development of a specific substrate-competitive inhibitor.

Specific regions of the kinase domain that are potentially good candidates for peptide targets for designing inhibitors molecules include, but are not limited to: the ATP-binding pocket between the N- and C-terminal kinase lobes (comprising amino acid residues 975-984, 1001-1003, 1033-1034, 1049-1056, 1010-1012, 1122-1123; see Appendix A); the peptide substrate binding groove in the C-terminal kinase lobe (comprising amino acid residues 1137-1157, 1105-1109; see Appendix A); the hinge region on the backside of the kinase domain, which is opposite the ATP-binding pocket (comprising amino acid residues 1025-1035, 1112-1121, 1050-1055; see Appendix A); and the alpha helix C (comprising amino acid residues 1005-1029; see Appendix A)

General Methods

In one embodiment of the invention, the three-dimensional structural information of an IGF1RK domain is used as a target in a virtual ligand screening procedure that seeks to identify, via computer docking methods, those candidate compounds in a vast compound library which are capable of binding to the target site with high affinity.

In another embodiment, the structural information of an IGF1RK domain is used to design compounds predicted to bind to the IGF1RK domain, and such compounds are tested for high affinity binding.

Compounds derived or obtained from either approach scoring the highest in the docking procedure are then tested in cell-based and cell-free assays (described below) to determine their efficacy in inhibiting IGF1 activity. In one embodiment of the invention, a compound identified by the instant methods blocks IGF1 activity by acting as an IGF1 antagonist. Alternatively, a compound identified by the instant methods may enhance IGF1 activity by acting as an agonist of IGF1-mediated signaling.

A compound identified using a method of the present invention may also be co-crystallized with IGF1RK to verify binding in the IGF1RK domain. In a further embodiment of the invention, candidate compounds (e.g., peptides) capable of binding to the IGF1RK domain are modified by methods known in the art to further improve specific characteristics, e.g., to increase efficacy and/or specificity and/or solubility.

As used herein, the term “modified peptide” may be used to refer to a peptide that is capable of binding to a protein and modulating its activity (e.g., a tyrosine kinase domain of a cell surface receptor). Modified peptides may possess features that, for example, modulate (increase or decrease) binding, alter the half-life of the peptide, decrease renal clearance, or improve absorption.

As described and discussed earlier herein, the term “amino acid” and any reference to a specific amino acid is also meant to include naturally occurring proteogenic amino acids as well as non-naturally occurring amino acids such as amino acid analogs. One of skill in the art would know that this definition includes, unless otherwise specifically indicated, naturally occurring proteogenic (D) or (L) amino acids, chemically modified amino acids, including amino acid analogs such as penicillamine (3-mercapto-D-valine), naturally occurring non-proteogenic amino acids such as norleucine and chemically synthesized compounds that have properties known in the art to be characteristic of an amino acid. As used herein, the term “proteogenic” indicates that the amino acid can be incorporated into a protein in a cell through well-known metabolic pathways.

The choice of including an (L)- or a (D)-amino acid into a peptide identified using a method of the present invention depends, in part, on the desired characteristics of the peptide. For example, the incorporation of one or more (D)-amino acids can confer enhanced peptide stability in vitro and/or in vivo. The incorporation of one or more (D)-amino acids can also increase or decrease the binding activity of the peptide as determined, for example, using the binding assays described herein, or other methods well known in the art. In some cases it is desirable to design a peptide which retains activity for a short period of time, for example, when designing a peptide to administer to a subject. In these cases, the incorporation of one or more (L)-amino acids into a peptide can promote digestion of the peptide by endogenous peptidases in a subject to whom the peptide has been administered. This feature serves to limit the subject's exposure to an active peptide.

As used herein, the term “amino acid equivalent” refers to compounds which depart from the structure of the naturally occurring amino acids, but which have substantially the structure of an amino acid, such that they can be substituted within a peptide which retains is biological activity. Thus, for example, amino acid equivalents can include amino acids having side chain modifications or substitutions, and related organic acids, amides or the like. The term “amino acid” is intended to include amino acid equivalents. The term “residues” refers both to amino acids and amino acid equivalents.

As used herein, the term “peptide” is used in its broadest sense to refer to compounds containing amino acid equivalents or other non-amino groups, while still retaining the desired functional activity of a peptide. Peptide equivalents can differ from conventional peptides by the replacement of one or more amino acids with related organic acids (such as PABA), amino acids or the like or the substitution or modification of side chains or functional groups.

It is to be understood that limited modifications can be made to a peptide without destroying its biological function. Thus, modified forms of peptides identified using a method of the invention are encompassed herein, as long as they retain an activity of the peptide. Modifications can include, for example, additions, deletions, or substitutions of amino acids residues, substitutions with compounds that mimic amino acid structure or functions, as well as the addition of chemical moieties such as amino or acetyl groups. The modifications can be deliberate or accidental, and can be modifications of the composition or the structure.

Selected compounds exhibiting desirable characteristics are designated lead compounds, and further tested in animal models to measure their efficacy.

Virtual Ligand Screening Via Flexible Docking Technology

Knowledge pertaining to a specific receptor structure can be used in conjunction with current docking and screening methodologies to select small sets of likely lead candidate ligands from large libraries of compounds. Such methods are described, for example, in Abagyan and Totrov (2001) Current Opinion Chemical Biology 5:375-382, herein specifically incorporated by reference in its entirety.

Virtual ligand screening (VLS) based on high-throughput flexible docking is useful for designing and identifying compounds capable of binding to a specific receptor structure. VLS can be used to virtually sample a large number of chemical molecules without synthesizing and experimentally testing each one. Generally, the methods start with receptor modeling which uses a selected receptor structure derived by conventional means, e.g., X-ray crystallography, NMR, homology modeling. A set of compounds and/or molecular fragments are then docked into the selected binding site using any one of the existing docking programs, such as for example, MCDOCK (Liu et al. (1999) J. Comput. Aided Mol. Des. 13:435-451), SEED (Majeux et al. (1999) Proteins 37:88-105; DARWIN (Taylor et al. (2000) Proteins 41:173-191; MM (David et al. (2001) J. Comput. Aided Mol. Des. 15:157-171. Compounds are scored as ligands, and a list of candidate compounds predicted to possess the highest binding affinities are generated for further in vitro and in vivo testing and/or chemical modification.

In one approach of VLS, molecules are “built” into a selected binding pocket prior to chemical generation. A large number of programs are designed to “grow” ligands atom-by-atom [see, for example, GENSTAR (Pearlman et al. L(1993) J. Comput. Chem. 14:1184), LEGEND (Nishibata et al. (1993) J. Med. Chem. 36:2921-2928), MCDNLG (Rotstein et al. (1993) J. Comput-Aided Mol. Des. 7:23-43), CONCEPTS (Gehlhaar et al. (1995) J. Med. Chem 38:466-472] or fragment-by-fragment [see, for example, GROUPBUILD (Rotsein et al. (1993) J. Med. Chem. 36:1700-1710), SPROUT (Gillet et al. (1993) J. Comput. Aided Mol. Des. 7:127-153), LUDI (Bohm (1992) J. Comput. Aided Mol. Des. 6:61-78), BUILDER (Roe (1995) J. Comput. Aided Mol. Des. 9:269-282), and SMOG (DeWitte et al. (1996) J. Am. Chem. Soc. 118:11733-11744].

Methods for scoring ligands for a particular receptor are known which allow discrimination between the small number of molecules able to bind the receptor structure and the large number of non-binders. See, for example, Agagyan et al. (2001) supra, for a report on the growing number of successful ligands identified via virtual ligand docking and screening methodologies.

For example, Nishibata et al. (1993) J. Med. Chem 36:2921-2928, describe the ability of a structure construction program to generate inhibitory molecules based on the three-dimensional structure of the active site of a molecule, dihydrofolate reductase. The program was able to predict molecules having a similar structure to four known inhibitors of the enzyme, providing strong support that new lead compounds can be obtained with knowledge pertaining to the three dimensional structure of the target. Similarly, Gillet et al. (1993) J. Computer Aided Mol. Design 7:127-153 describe structure generation through artificial intelligence techniques based on steric constrains (SPROUT).

The invention provides methods for identifying agents (e.g., candidate compounds or test compounds) that bind with high affinity to the IGF1RK domain. Agents identified by the screening method of the invention are useful as candidate anti-cancer therapeutics, and/or in any condition which could be ameliorated by inhibition of the tyrosine kinase activity of the IGF1 receptor. Such conditions include, but are not limited to a diabetic complication exacerbated by IGF-1, acromegaly, age-related macular degeneration, ischemic injury, and trauma.

Malignant transformation is often associated with increased expression and/or constitutive activation of the IGF-1R. Indeed, aberrant signaling of the IGF1R has been implicated in a variety of cancers, including: multiple myeloma, lymphatic metastasis, lung cancer (e.g., carcinoma), breast cancer, Wilms' tumor, cervical cancer, prostate cancer, colorectal cancer, glioma, and rhabdomyosarcoma (RMS).

Agents Identified by the Screening Methods of the Invention

The invention provides methods for identifying agents (e.g., candidate compounds or test compounds) that bind with high affinity to the IGF1RK domain. Agents identified by the screening methods of the invention may be used as candidate therapeutics for hyperproliferative disorders, such as, for example, cancer.

Examples of agents, candidate compounds or test compounds include, but are not limited to, nucleic acids (e.g., DNA and RNA), carbohydrates, lipids, proteins, peptides, peptidomimetics, small molecules and other drugs. Agents can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds (Lam (1997) Anticancer Drug Des. 12:145; U.S. Pat. No. 5,738,996; and U.S. Pat. No. 5,807,683, each of which is incorporated herein in its entirety by reference).

Examples of methods for the synthesis of molecular libraries can be found in the art, for example in: DeWitt et al. (1993) Proc. Natl. Acad. Sci. USA 90:6909; Erb et al. (1994) Proc. Natl. Acad. Sci. USA 91:11422; Zuckermann et al. (1994) J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and Gallop et al. (1994) J. Med. Chem. 37:1233, each of which is incorporated herein in its entirety by reference.

Libraries of compounds may be presented, e.g., presented in solution (e.g., Houghten (1992) Bio/Techniques 13:412-421), or on beads (Lam (1991) Nature 354:82-84), chips (Fodor (1993) Nature 364:555-556), bacteria (U.S. Pat. No. 5,223,409), spores (U.S. Pat. Nos. 5,571,698; 5,403,484; and 5,223,409), plasmids (Cull et al. (1992) Proc. Natl. Acad. Sci. USA 89:1865-1869) or phage (Scott and Smith (1990) Science 249:386-390; Devlin (1990) Science 249:404-406; Cwirla et al. (1990) Proc. Natl. Acad. Sci. USA 87:6378-6382; and Felici (1991) J. Mol. Biol. 222:301-310), each of which is incorporated herein in its entirety by reference.

Screening Assays

Small molecules identified through the above described virtual ligand docking and screening methodologies are further tested in in vitro and in vivo assays. In one embodiment, agents that interact with (i.e., bind to) the IGF1RK domain of the IGF1 receptor, or a functional fragment thereof, are identified and/or confirmed in a cell-free assay system. In accordance with this embodiment, a native or recombinant IGF1 receptor or fragment thereof is contacted with a candidate compound or a control compound and the ability of the candidate compound to interact with the IGF1 RK domain of the IGF1 receptor is determined. If desired, this assay may be used to screen a plurality (e.g. a library) of candidate compounds. In one embodiment, the IGF1 receptor or fragment thereof is first immobilized by contacting it with, for example, an immobilized antibody which specifically recognizes and binds to it, or by contacting a purified preparation of the IGF1 receptor or fragment thereof, with a surface designed to bind proteins. The IGF1 receptor or IGF1RK domain-containing IGF1 receptor fragment may be partially or completely purified (e.g., partially or completely free of other polypeptides) or part of a cell lysate. Further, the IGF1 receptor or IGF1RK domain-containing IGF1 receptor fragment may be a fusion protein comprising the IGF1RK domain or a biologically active portion thereof, and a domain such as glutathionine-S-transferase. Alternatively, the IGF1 receptor or IGF1RK-containing fragment thereof can be biotinylated using techniques well known to those of skill in the art (e.g., biotinylation kit, Pierce Chemicals; Rockford, Ill.). The ability of a candidate compound to interact with the IGF1RK domain can be determined by methods known to those of skill in the art.

In Vitro Assays

The present inventors have devised methods to identify small molecule inhibitors for the unphosphorylated form of IGF1 receptor (0P) and the fully-activated, triply-phosphorylated form of IGF1 receptor (3P). For both forms of the enzyme, a continuous spectrophotometric kinase assay is used. In this assay, production of ADP is coupled to the oxidation of NADH, which is measured as a decrease in absorbance at 340 nm. Using this method, the autophosphorylation of 0P is measured directly, rather than indirectly via substrate phosphorylation. The method may be used in a microtiter plate format, for example, to screen for modulators (i.e., inhibitors or activators) of the phosphorylation/dephosphorylation rate of these different forms of the IGF1R and/or substrates thereof. Inhibitors, for example, that decrease the rate of 0P autophosphorylation can be identified using this method. In the case of 3P, the reactions may also comprise a synthetic peptide substrate, and the ability of inhibitory candidates to block peptide phosphorylation may be measured.

In one embodiment, the assays are performed in 100 mM Tris-HCl (pH 7.5), 10 mM MgCl₂, 1 mM phosphoenolpyruvate, 0.28 mM NADH, 89 units/ml pyruvate kinase, 124 units/ml lactate dehydrogenase, 2% DMSO, and at 30° C. in a 50 microliter reaction volume. Reactions are initiated by the addition of ATP to mixtures containing enzyme and various concentrations of inhibitors. Assays of IGF1RK-0P autophosphorylation may be performed, for example, at 6 micromolar enzyme and 1 mM ATP. The IGF1RK-3P peptide phosphorylation assays are generally carried out with 150 nM enzyme, 100 micromolar ATP, and 50 micromolar peptide substrate (KKEEEEYMMMM; SEQ ID NO: 2).

Cell Based Assays

In another embodiment, agents that interact with (i.e., bind to) the IGF1RK domain are tested in a cell-based assay system. In accordance with this embodiment, cells expressing an IGF1 receptor or a fragment thereof containing the IGF1RK domain, are contacted with a candidate compound or a control compound and the ability of the candidate compound to interact with the IGF1 receptor is determined. If desired, this assay may be used to screen a plurality (e.g. a library) of candidate compounds. A cell, for example, can be of prokaryotic origin (e.g., E. coli) or eukaryotic origin (e.g., yeast or mammalian). Further, the cells can express the IGF1 receptor, or functional IGF1 receptor fragment, endogenously or be genetically engineered to express the IGF1 receptor, or functional fragment thereof. In certain instances, the IGF1 receptor or functional fragment thereof is labeled, for example with a radioactive label (such as ³²P, ³⁵S or ¹²⁵I) or a fluorescent label (such as fluorescein isothiocyanate, rhodamine, phycoerythrin, phycocyanin, allophycocyanin, o-phthaldehyde or fluorescamine) to enable detection of an interaction between the IGF1 receptor and a candidate compound. The ability of the candidate compound to bind to the IGF1RK domain can be determined by methods known to those of skill in the art. For example, the interaction between a candidate compound and the IGF1RK domain can be determined by flow cytometry, a scintillation assay, immunoprecipitation or western blot analysis.

In general, IGF1R-expressing cells are treated with modulators (i.e., inhibitors or activators) of IGF1R activity and cell lysates are generated from these treated cells. IGF1Rs are isolated from the lysate by immunoprecipitation and analyzed for phosphotyrosine content. Alternatively, or in addition, protein substrates known to be phosphorylated by IGFR may be immunoprecipitated from the cellular lysate and their phosphotyrosine content determined.

Chinese Hamster Ovary (CHO) cells overexpressing wild type IGF-1 receptors are an exemplary cellular model in which to perform such assays. Such cells may be maintained in DMEM supplemented with 10% dialyzed FBS, 100 μM non essential amino acids, 1% L-glutamine, 1% antibiotic and antimycotic solution, 500 μg/ml Geneticin, and 2 μM methotrexate in a humidified atmosphere of 95% air and 5% CO₂ at 37° C. Confluent cells in 60-mm plates are incubated overnight with serum-free (SF) medium (HAM F-12, 0.5% sterile BSA, and 1% antibiotic and antimycotic). Inhibitors are subsequently added at various concentrations in fresh SF medium for 1 hour. Cells are then stimulated with 10 nM IGF-1 for 1 minute. After treatment, cells are washed twice with ice-cold PBS, harvested, and lysed with fresh lysis buffer (25 mM Tris pH 8.0, 2 mM EDTA pH 8.0, 140 mM NaCl, 1% NP-40, 10 μg/ml aprotinin, 20 μM phenylmethylsulphonyl fluoride (PMSF), 10 μg/ml Leupeptin and 10 mM orthovanadate) for 1 hour at 4° C., after which the lysates are cleared by centrifugation at 12,000×g for 10 minutes. Protein concentrations of the postnuclear supernatants are determined by the Bradford method (Bio-Rad). To measure tyrosine phosphorylation of the β-subunits of the IGF-1 receptors, lysates are incubated overnight at 4° C. with 2 μg of anti-IGF1R antibody (C-20; Santa Cruz Biotechnology) and 50 μl of 50% protein-A agarose slurry. After 3 washes with lysis buffer, pellets are resuspended in SDS-PAGE sample buffer and boiled for 3 minutes. Proteins are resolved by SDS-PAGE (7.5%) and transferred by electroblotting onto PVDF membranes. Tyrosine phosphorylated receptors are detected by immunoblotting with anti-phosphotyrosine antibody (4G10, Upstate Biotechnology) and then stripped and reprobed with anti-IGF1R antibody. Detection was with the ECL method (Amersham). As indicated above, parallel experiments may be performed in which IGFR substrate are specifically immunoprecipitated, immunoblotted, and probed with anti-phosphotyrosine antibody.

In another embodiment, agents that modulate (i.e., decrease or increase) the activity of IGF1 receptor kinase activity are identified/confirmed in an animal model. Examples of suitable animals include, but are not limited to, mice, rats, rabbits, monkeys, guinea pigs, dogs and cats. Preferably, the animal used provides an animal model system for a hyperproliferative disorder associated with altered or aberrant IGF1 receptor activity. In accordance with this embodiment, the test compound or a control compound is administered (e.g., orally, rectally or parenterally such as intraperitoneally or intravenously) to a suitable animal and the effect on the level of IGF1 receptor kinase activity is determined.

Exemplary Methods of Use for the IGF1RK Domain Binding Agents

The invention provides for treatment of disorders ameliorated by administration of a therapeutic compound identified using the method of the invention. Such compounds include but are not limited to proteins, peptides, protein or peptide derivatives or analogs, antibodies, nucleic acids, and small molecules.

The invention provides methods for treating (therapeutically and prophylactically) IGF1-related hyperproliferative disorders (e.g., cancer), comprising administering to a subject an effective amount of a compound identified by a method of the invention. In a preferred aspect, the compound is substantially purified (e.g., substantially free from substances that limit its effect or produce undesired side-effects). The subject is preferably an animal, including but not limited to animals such as cows, pigs, horses, chickens, cats, dogs, etc., and is preferably a mammal, and most preferably human. In a specific embodiment, a non-human mammal is the subject.

As used herein, the term “treating” refers to both therapeutic treatment and prophylactic or preventative measures. Those in need of treatment include those subjects or patients exhibiting symptoms of the disorder, as well as those subjects that are predisposed to the disorder, or diagnosed with the disorder in the absence of symptoms (asymptomatic patients), or in whom the disorder is to be prevented. Consecutive treatment or administration refers to treatment on at least a daily basis without interruption for one or more days. Intermittent treatment or administration, or treatment or administration in an intermittent fashion, refers to treatment that is not consecutive, but rather cyclic in nature. The treatment regime herein can be either consecutive or intermittent. Subjects for whom the preventive measures are appropriate include those with one or more known risk factors for an IGF1R-related disorder, such as cancer.

A “disorder” is any condition caused, mediated, exacerbated by, or associated with IGF1 receptor activity that would benefit from treatment with modulators identified using the methods of the present invention. Such conditions include chronic and acute disorders, or pathological conditions that predispose a mammal to a particular disorder. Non-limiting examples of disorders to be treated herein include diseases associated with undesirable cell proliferation, such as benign tumors, cancer, restenosis, and asthma; acromegaly; inflammatory, angiogenic, or immunological disorders; an ischemic injury such as a stroke, myocardial ischemia, or ischemic injury to the kidneys; diabetic complications such as diabetic retinopathies or neuropathies; eye-related diseases; or neuronal, glial, astrocyte-related, hypothalamic or other glandular, macrophage, epithelial, or stromal disorders. Eye-related disorders include age-related macular degeneration; ophthalmic surgery such as cataract extraction, corneal transplantation, glaucoma filtration surgery, and keratoplasty; surgery to correct refraction, i.e., a radial keratotomy, also in sclera macular holes and degeneration; retinal tears; vitreoretinopathy; cataract disorders of the cornea such as the sequelae of radial keratotomy; dry eye; viral conjunctivitis; ulcerative conjunctivitis; optical wounds such as corneal epithelial wounds; Sjogren's syndrome; macular and retinal edema; vision-limited scarring; and retinal ischemia. Preferably, such disorders are cancer, a diabetic complication, an ischemic injury, acromegaly, restenosis, an eye-related disorder, or asthma. The efficacy of the treatment can be evidenced by a reduction in clinical manifestations or symptoms, including, for example, decreased cell proliferation or growth, improved renal clearance, improved vision, or a reduction in the amount of IGF receptor signaling.

The term “effective amount” refers to an amount of a small molecule modulator effective to treat a disease or disorder in a mammal. In the case of cancer, the effective amount of a modulator (i.e., an inhibitor) may reduce the number of cancer cells; reduce tumor size; reduce cancer cell infiltration into peripheral organs; reduce tumor metastasis; and/or relieve one or more of the symptoms associated with the disorder. In cancer therapy, for example, in vivo efficacy can be measured by assessing the time to disease progression (TTP) and/or determining the response rate (RR).

The terms “cancer” and “cancerous” refer to or describe a physiological condition, generally observed in mammals, that is characterized by unregulated cell growth. Examples of cancer include but are not limited to, carcinoma, lymphoma, blastoma, sarcoma, and leukemia. More particular examples of such cancers include squamous cell cancer, lung cancer (including small-cell lung cancer, non-small cell lung cancer, adenocarcinoma of the lung, and squamous carcinoma of the lung), cancer of the peritoneum, hepatocellular cancer, gastric or stomach cancer (including gastrointestinal cancer), pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney or renal cancer, liver cancer, prostate cancer, vulval cancer, thyroid cancer, hepatic carcinoma and various types of head and neck cancer, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); Hairy cell leukemia; chronic myeloblastic leukemia; and post-transplant lymphoproliferative disorder (PTLD). Preferably, the cancer comprises a tumor that expresses an IGF receptor, more preferably breast cancer, lung cancer, colorectal cancer, or prostate cancer, and most preferably breast or prostate cancer.

Formulations and methods of administration that can be employed when the compound comprises a nucleic acid are described above; additional appropriate formulations and routes of administration are described below.

Various delivery systems are known and can be used to administer a compound of the invention, e.g., encapsulation in liposomes, microparticles, microcapsules, recombinant cells capable of expressing the compound, receptor-mediated endocytosis (see, e.g., Wu and Wu (1987) J. Biol. Chem. 262:4429-4432), construction of a nucleic acid as part of a retroviral or other vector, etc. Methods of introduction can be enteral or parenteral and include but are not limited to intradermal, intramuscular, intraperitoneal, intravenous, subcutaneous, intranasal, epidural, and oral routes. The compounds may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce the pharmaceutical compositions of the invention into the central nervous system (CNS) by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

In a specific embodiment, it may be desirable to administer the pharmaceutical compositions of the invention locally, e.g., by local infusion during surgery, topical application, e.g., by injection, by means of a catheter, or by means of an implant, said implant being of a porous, non-porous, or gelatinous material, including membranes, such as sialastic membranes, or fibers. In one embodiment, administration can be by direct injection into cerebrospinal fluid (CSF) or at the site (or former site) of hyperproliferative cells in CNS tissue.

In another embodiment, the compound can be delivered in a vesicle, in particular a liposome (see Langer (1990) Science 249:1527-1533; Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.).

In yet another embodiment, the compound can be delivered in a controlled release system. In one embodiment, a pump may be used (see Langer, supra; Sefton (1987) CRC Crit. Ref. Biomed. Eng. 14:201; Buchwald et al. (1980) Surgery 88:507; Saudek et al., 1989, N. Engl. J. Med. 321:574). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, J., 1983, Macromol. Sci. Rev. Macromol. Chem. 23:61; see also Levy et al. (1985) Science 228:190; During et al. (1989) Ann. Neurol. 25:351; Howard et al. (1989) J. Neurosurg. 71:105). In yet another embodiment, a controlled release system can be placed in proximity of the therapeutic target, e.g., an IGF receptor expressing tumor, thus requiring only a fraction of the systemic dose (see, e.g., Goodson, in Medical Applications of Controlled Release, supra, vol. 2, pp. 115-138 (1984)).

Other controlled release systems are discussed in the review by Langer (1990, Science 249:1527-1533).

Pharmaceutical Compositions

The present invention also provides pharmaceutical compositions. Such compositions comprise a therapeutically effective amount of an agent, and a pharmaceutically acceptable carrier. In a particular embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the compound, preferably in purified form, together with a suitable amount of carrier so as to provide the form for proper administration to the subject. The formulation should suit the mode of administration.

In a preferred embodiment, the composition is formulated in accordance with routine procedures as a pharmaceutical composition adapted for intravenous administration to a human. Typically, compositions for intravenous administration are solutions in sterile isotonic aqueous buffer. Where necessary, the composition may also include a solubilizing agent and a local anesthetic such as lidocaine to ease pain at the site of the injection. Generally, the ingredients are supplied either separately or mixed together in unit dosage form, for example, as a dry lyophilized powder or water free concentrate in a hermetically sealed container such as an ampoule or sachet indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compounds of the invention can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

The amount of a compound of the invention which will be effective in the treatment of a hyperproliferative disorder can be determined by standard clinical techniques based on the present description. In addition, in vitro assays may optionally be employed to help identify optimal dosage ranges. The precise dose to be employed in the formulation will also depend on the route of administration, and the seriousness of the disease or disorder, and should be decided according to the judgment of an attending physician and the patient's condition. However, suitable dosage ranges for intravenous administration are generally about 20-500 micrograms of active compound per kilogram body weight. Suitable dosage ranges for intranasal administration are generally about 0.01 pg/kg body weight to 1 mg/kg body weight. Effective doses may be extrapolated from dose-response curves derived from in vitro or animal model test systems.

Suppositories generally contain active ingredient in the range of 0.5% to 10% by weight; oral formulations preferably contain 10% to 95% active ingredient.

Nucleic Acids

The invention provides methods for identifying agents capable of binding the IGF1RK domain to modulate (i.e., inhibit or activate) tyrosine kinase activity of the IGF1 receptor. Accordingly, the invention encompasses administration of a nucleic acid encoding a peptide or protein modulator of the tyrosine kinase domain.

In one embodiment, a nucleic acid comprising a sequence encoding a peptide or protein capable of inhibiting the IGF1RK domain of the IGF1 receptor is administered. Any suitable methods for administering a nucleic acid sequence available in the art can be used according to the present invention.

In an alternate embodiment, a nucleic acid comprising a sequence encoding a peptide or protein capable of activating the IGF1RK domain of the IGF1 receptor or maintaining the IGF1 receptor in an activated state or conformation is administered. Any suitable methods for administering a nucleic acid sequence known in the art can be used according to the present invention.

Methods for administering and expressing a nucleic acid sequence are generally known in the area of gene therapy. For general reviews of the methods of gene therapy, see Goldspiel et al. (1993) Clinical Pharmacy 12:488-505; Wu and Wu (1991) Biotherapy 3:87-95; Tolstoshev (1993) Ann. Rev. Pharmacol. Toxicol. 32:573-596; Mulligan (1993) Science 260:926-932; and Morgan and Anderson (1993) Ann. Rev. Biochem. 62:191-217; May (1993) TIBTECH 11(5): 155-215. Methods commonly known in the art of recombinant DNA technology that can be used in the present invention are described in Ausubel et al. (eds.), 1993, Current Protocols in Molecular Biology, John Wiley & Sons, NY; and Kriegler (1990) Gene Transfer and Expression, A Laboratory Manual, Stockton Press, NY.

In a particular aspect, a compound comprises a nucleic acid encoding a peptide or protein capable of competitively binding to the IGF1RK domain of the IGF1 receptor and inhibiting its tyrosine kinase activity, such nucleic acid being part of an expression vector that expresses the peptide or protein in a suitable host. In particular, such an expression vector has a promoter operably linked to the coding region, said promoter being inducible or constitutive (and, optionally, tissue-specific). In another particular embodiment, a nucleic acid molecule is used in which the coding sequences and any other desired sequences are flanked by regions that promote homologous recombination at a desired site in the genome, thus providing for intrachromosomal expression of the nucleic acid (Koller and Smithies (1989) Proc. Natl. Acad. Sci. USA 86:8932-8935; Zijlstra et al. (1989) Nature 342:435-438).

Delivery of the nucleic acid into a subject may be direct, in which case the subject is directly exposed to the nucleic acid or nucleic acid-carrying vector; this approach is known as in vivo gene therapy. Alternatively, delivery of the nucleic acid into the subject may be indirect, in which case cells are first transformed with the nucleic acid in vitro and then transplanted into the subject, known as “ex vivo gene therapy”.

In another embodiment, the nucleic acid is directly administered in vivo, where it is expressed to produce the encoded product. This can be accomplished by any of numerous methods known in the art, e.g., by constructing it as part of an appropriate nucleic acid expression vector and administering it so that it becomes intracellular, e.g., by infection using a defective or attenuated retroviral or other viral vector (see U.S. Pat. No. 4,980,286); by direct injection of naked DNA; by use of microparticle bombardment (e.g., a gene gun; Biolistic, Dupont); by coating with lipids, cell-surface receptors or transfecting agents; by encapsulation in liposomes, microparticles or microcapsules; by administering it in linkage to a peptide which is known to enter the nucleus; or by administering it in linkage to a ligand subject to receptor-mediated endocytosis (see, e.g., Wu and Wu, 1987, J. Biol. Chem. 262:4429-4432), which can be used to target cell types specifically expressing the receptors. In another embodiment, a nucleic acid-ligand complex can be formed in which the ligand comprises a fusogenic viral peptide that disrupts endosomes, allowing the nucleic acid to avoid lysosomal degradation. In yet another embodiment, the nucleic acid can be targeted in vivo for cell specific uptake and expression, by targeting a specific receptor (see, e.g., PCT Publications WO 92/06180 dated Apr. 16, 1992 (Wu et al.); WO 92/22635 dated Dec. 23, 1992 (Wilson et al.); WO92/20316 dated Nov. 26, 1992 (Findeis et al.); WO93/14188 dated Jul. 22, 1993 (Clarke et al.), WO 93/20221 dated Oct. 14, 1993 (Young)). Alternatively, the nucleic acid can be introduced intracellularly and incorporated within host cell DNA for expression, by homologous recombination (Koller and Smithies, 1989, Proc. Natl. Acad. Sci. USA 86:8932-8935; Zijlstra et al. (1989) Nature 342:435-438).

In a further embodiment, a retroviral vector can be used (see Miller et al. (1993) Meth. Enzymol. 217:581-599). These retroviral vectors have been modified to delete retroviral sequences that are not necessary for packaging of the viral genome and integration into host cell DNA. The nucleic acid encoding the protein to be used in gene therapy is cloned into the vector, which facilitates delivery of the gene into a subject. More detail about retroviral vectors can be found in Boesen et al. (1994) Biotherapy 6:291-302, which describes the use of a retroviral vector to deliver the mdr1 gene to hematopoietic stem cells in order to make the stem cells more resistant to chemotherapy. Other references illustrating the use of retroviral vectors in gene therapy are: Clowes et al. (1994) J. Clin. Invest. 93:644-651; Kiem et al. (1994) Blood 83:1467-1473; Salmons and Gunzberg (1993) Human Gene Therapy 4:129-141; and Grossman and Wilson (1993) Curr. Opin. in Genetics and Devel. 3:110-114.

Other viral vectors, including adenoviruses, may be used in gene therapy. Adenoviruses are especially attractive vehicles for delivering genes to respiratory epithelia. Adenoviruses naturally infect respiratory epithelia, the infection of which results in a mild respiratory disease. Other targets for adenovirus-based delivery systems are the liver, central nervous system, endothelial cells, and muscle. Moreover, adenoviruses have the advantage of being capable of infecting non-dividing cells. Kozarsky and Wilson (1993) Current Opinion in Genetics and Development 3:499-503 present a review of adenovirus-based gene therapy. Bout et al. (1994) Human Gene Therapy 5:3-10 demonstrated the utility of adenovirus vectors for introducing genes into the respiratory epithelia of rhesus monkeys. Other instances pertaining to the use of adenoviruses in gene therapy can be found in Rosenfeld et al. (1991) Science 252:431-434; Rosenfeld et al. (1992) Cell 68:143-155; Mastrangeli et al. (1993) J. Clin. Invest. 91:225-234; PCT Publication WO94/12649; and Wang, et al. (1995) Gene Therapy 2:775-783. Adeno-associated virus (AAV) has also been proposed for use in gene therapy (Walsh et al. (1993) Proc. Soc. Exp. Biol. Med. 204:289-300; U.S. Pat. No. 5,436,146).

Another suitable approach to gene therapy involves transferring a gene to cells in tissue culture by methods such as, for example, viral infection or electroporation-mediated, liposome-mediated, or calcium phosphate-mediated transfection. Usually, the method of transfer also includes the transfer of a selectable marker into the cells. The cells are then placed under selection to isolate those cells that have been productively transfected. Such selected cells are then delivered to a subject.

In this embodiment, the nucleic acid is introduced into a cell prior to administration of the resulting recombinant cell in vivo. Such introduction can be carried out by any method known in the art, including, but not limited to, transfection, microinjection, infection with a viral or bacteriophage vector comprising the desired nucleic acid sequences, cell fusion, chromosome-mediated gene transfer, microcell-mediated gene transfer, and spheroplast fusion. Numerous techniques are known in the art for the introduction of foreign genes into cells (see, e.g., Loeffler and Behr (1993) Meth. Enzymol. 217:599-618; Cohen et al. (1993) Meth. Enzymol. 217:618-644; Cline (1985) Pharmac. Ther. 29:69-92) and may be used in accordance with the present invention, provided that the necessary developmental and physiological functions of the recipient cells are not disrupted. Such a technique provides for the stable transfer of the nucleic acid to the cell, so that the nucleic acid is expressible by the cell and preferably heritable and expressible by its cell progeny.

The resulting recombinant cells can be delivered to a subject by various methods known in the art. In a preferred embodiment, epithelial cells are injected, e.g., subcutaneously. In another embodiment, recombinant skin cells may be applied as a skin graft onto the subject; recombinant blood cells (e.g., hematopoietic stem or progenitor cells) are preferably administered intravenously. The amount of cells envisioned for use depends on the desired effect, the condition of the subject, etc., and can be determined by one skilled in the art.

Cells into which a nucleic acid can be introduced for purposes of gene therapy encompass any desired, available cell type, and include but are not limited to hepatocyte cells, muscle cells, glial cells (e.g., oligodendrocytes or astrocytes), epithelial cells, endothelial cells, keratinocytes, and fibroblasts; blood cells such as T lymphocytes, B lymphocytes, monocytes, macrophages, neutrophils, eosinophils, megakaryocytes, granulocytes; various stem or progenitor cells, in particular hematopoietic stem or progenitor cells, e.g., as obtained from bone marrow, umbilical cord blood, peripheral blood or fetal liver. In a preferred embodiment, a cell used for gene therapy is autologous to the treated subject.

In an embodiment in which recombinant cells are used in gene therapy, a nucleic acid encoding an agent (e.g., a peptide or protein) capable of modulating the activity of an IGF1 receptor is introduced into cells such that it is expressible by the cells and/or their progeny, and the recombinant cells are then administered in vivo for therapeutic effect. In a specific embodiment, stem or progenitor cells are used. Any stem or progenitor cells which can be isolated and maintained in vitro can be used in accordance with this embodiment of the present invention (see e.g. PCT Publication WO 94/08598, dated Apr. 28, 1994; Stemple and Anderson (1992) Cell 71:973-985; Rheinwald (1980) Meth. Cell Bio. 21A:229; and Pittelkow and Scott (1986) Mayo Clinic Proc. 61:771).

In another embodiment, the nucleic acid to be introduced for purposes of gene therapy may comprise an inducible promoter operably linked to the coding region, such that expression of the nucleic acid is controllable by regulating the presence or absence of the appropriate inducer of transcription.

Direct injection of a DNA coding for a peptide or protein capable of binding to the IGF1RK domain of the IGF1 receptor and modulating its activity may also be performed according to, for example, the techniques described in U.S. Pat. No. 5,589,466. These techniques involve the injection of “naked DNA”, i.e., isolated DNA molecules in the absence of liposomes, cells, or any other material besides a suitable carrier. The injection of DNA encoding a protein and operably linked to a suitable promoter results in the production of the protein in cells near the site of injection and potentially the elicitation of an immune response in the subject to the protein encoded by the injected DNA.

Kits

The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects (a) approval by the agency of manufacture, use or sale for human administration, (b) directions for use, or both.

### EXAMPLES

The following examples are set forth so as to provide those of ordinary skill in the art with a complete disclosure and description of how to make and use the assay, screening, and therapeutic methods of the invention, and are not intended to limit the scope of the invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1

### Expression and Purification of IGF1RK

Spodoptera frugiperda (Sf9) cells were infected with a recombinant baculovirus encoding residues 956-1,256 of the human insulin-like growth factor 1 receptor (IGF1RK). Cells were harvested 72 hours post-infection and lysed in a French pressure cell in 20 mM Tris-HCl (pH 7.5), 5 mM EDTA, 2 mM DTT, 0.2% Triton X-lOO, 5 μg/ml leupeptin, 5 μg/ml aprotinin, 2 mM phenylmethylsulfonyl fluoride. Lysates were centrifuged at 10,000×g and filtered through a Millex 0.8 μm filter. IGF1RK was purified in three chromatographic steps on an FPLC system (Amersham Pharmacia Biotech): (1) Source-Q15, equilibrated in 20 mM Tris (pH 7.5), 1 mM DTT and eluted with a linear gradient of 1 M NaCl; (2) Superdex-75, developed in 20 mM Tris-HCl (pH 7.5), 0.15 M NaCl, and (3) Mono-Q HR 5/5, equilibrated in 20 mM Tris (pH 7.5) and eluted with a linear gradient from 0.1 M to 0.25 M NaCl over 60 ml.

To produce IGF1RK-3P, unphosphorylated IGF1RK was incubated with 10 mM ATP and 30 mM MgCl₂ for 5 minutes at room temperature. The autophosphorylation reaction was terminated by the addition of 100 mM EDTA, and then the reaction mixture was passed over a Superdex-75 gel filtration column. Sodium orthovanadate (200 μM) was added to inhibit any trace quantities of phosphatases, and the pooled fractions were diluted 1:2 in Mono-Q buffer. The four forms of IGF1RK were then separated on a Mono-Q column equilibrated in 20 mM Tris (pH 7.5) and eluted with a linear gradient from 0.1 M to 0.25 M NaCl over 60 ml. The buffer was exchanged with 20 mM Tris-HCl (pH 7.5) on an Ultra-free15 30K NMWL centrifugal filter device (Millipore) and the protein was concentrated to 10-15 mg/ml by selective filtration using a Centricon-30 filter (Amicon). After the final column, the protein was visualized as a single band by SDS-PAGE analysis. The 0P, 1P and 2P forms of IGF1RK were pooled individually after the Mono-Q column, concentrated, and stored at 4° C. in 20 mM Tris-HCl (pH 7.5). Final protein concentrations were determined by Bradford assay (Bio-Rad).

### Example 2

### Kinetic Analyses

Kinetic parameters for the four forms of IGF1RK were determined using a continuous spectrophotometric assay as described by Barker et al. (1995) Biochemistry 34: 14843-14851 and Porter et al. (2000) J. Biol. Chem. 275:2721-2726. All experiments were carried out at 30° C. in 50 μL of buffer containing 100 mM Tris (pH 7.5), 10 mM MgCl₂, 1 mM phosphoenolpyruvate, 0.2 mg/ml NADH, 111 units/ml pyruvate kinase, and 156 units/ml lactate dehydrogenase (Sigma). For determinations of Km for a peptide, reactions contained 50 nM enzyme, 1 mM ATP, and 0-2500 μM of the synthetic peptide substrate KKEEEEYMMMMG(SEQ ID NO: 2) (Songyang et at. (1995) Nature 373:536-539). For determinations of Km for ATP, reactions contained 250 nM enzyme, 2 mM peptide, and 0-2500 μM ATP. Kinetic parameters were determined by fitting data to the Michaelis-Menten equation. The conditions of the continuous assay (initial rate measurements, no buildup of ADP, 50 nM enzyme), do not promote IGF1RK dephosphorylation (Gruppuso et al. (1992) Biochem. Biophys. Res. Comm. 189:1457-1463; Al-Hasani et al. (1994) FEBS Lett. 349:17-22). Rates of ATP consumption for enzyme alone (<10% of rates with peptide) were subtracted before calculating kinetic constants. The kinetic results were confirmed using [γ-³²P]ATP and the phosphocellulose binding assay (results for IGF1RK-3P: Km for ATP=90.1±7.2 μM; Km for peptide=150±1:8 μM; Vₘₐₓ=8.2 μmol/min/mg).

### Example 3

### Crystallization of IGF1RK-3P

Crystals were grown at 4° C. by vapor diffusion in hanging drops containing 1.0 μl of protein solution (10 mg/ml IGF1RK-3P, 1 mM Mg AMP-PCP, 1 mM Y895 peptide) and 1.0 μl of reservoir solution [16% (w/v) polyethylene glycol (PEG) 8000, 100 mM HEPES pH 7.5, 0.15 M NaCl, and 2% (w/v) ethylene glycol]. The crystals belong to centered orthorhombic space group C222₁ and have unit cell dimensions a=80.6 Å, b=111.0 Å, and c=93.2 Å. There is one molecule in the asymmetric unit and the solvent content is 58% (assuming a partial specific volume of 0.74 cm3/g). AMP-PCP was purchased from Sigma Chemicals.

### Example 4

### Data Collection, Structure Determination and Analysis

One cryo-cooled crystal was used for data collection. The crystal was transferred into cryo-protectant equilibrated at 4° C. containing 20% (w/v) PEG 8000, 0.1 M HEPES (pH 7.5), 0.15 M NaCl, and 15% (w/v) ethylene glycol. Crystals were flash-cooled in liquid propane and transferred to a goniostat and cooled in a dry nitrogen stream at 100° K. Data were collected at beamline X4A at the National Synchrotron Light Source, Brookhaven National Laboratory, equipped with an ADSC Quantum-4 CCD detector. Data were processed using DENZO and SCALEPACK. A molecular replacement solution was found using AmoRe (Navaza (1994) Acta Crystallogr D 50:157-163) using a homology model generated by SWISS-MODEL (Peitsch (1996) Biochem Soc Trans 24:274-279) from Protein Data Bank entry 11R3. Rigid-body, positional, and B-factor refinements were carried out using CNS (Brunger et al. (1998) Acta Crystallogr D 54:905-921). Model building was performed using O (Jones et al. (1991) Acta Crystallogr A 47:110-119), all as described in Favelyukis et al. (2001) supra.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

1. A method for selecting a compound capable of binding to a tyrosine kinase domain of an insulin-like growth factor-1 (IGF1) receptor, comprising:
(a) determining an ability of a test compound to fit into a three-dimensional structure formed by the tyrosine kinase domain of the IGF1 receptor; and
(b) selecting a test compound predicted to fit the three-dimensional structure.

2. The method of claim 1, wherein the method is computer-assisted.

3. The method of claim 1, wherein the three-dimensional structure is the tyrosine kinase domain of the IGF1 receptor described by the coordinates of APPENDIX A.

4. The method of claim 1, wherein the IGF1 receptor comprises the sequence of SEQ ID NO: 1.

5. The method of claim 3, wherein the tyrosine kinase domain of the IGF1 receptor comprises amino acid residues 992-1292 of SEQ ID NO: 1.

6. The method of claim 2, wherein the computer-assisted method comprises virtual ligand docking and screening techniques capable of designing and/or identifying a compound predicted to bind a three-dimensional motif of the tyrosine kinase domain of the IGF1 receptor.

7. The method of claim 6, wherein the three-dimensional motif of the tyrosine kinase domain of the IGF1 receptor is selected from the group consisting of an ATP-binding pocket, a peptide substrate binding groove, a hinge region on the backside of the kinase domain, and an alpha helix C.

8. The method of claim 6, wherein the compound predicted to bind to a three-dimensional motif of the tyrosine kinase domain of the IGF1 receptor is predicted to bind with high affinity.

9. The method of claim 6, wherein binding to a three-dimensional motif of the tyrosine kinase domain of the IGF1 receptor is predicted to modulate an activity of the IGF1 receptor.

10. The method of claim 9, wherein modulating an activity of the IGF1 receptor reduces or inhibits an activity of the IGF1 receptor.

11. The method of claim 9, wherein modulating an activity of the IGF1 receptor increases or prolongs an activity of the IGF1 receptor.

12. A method of discriminating between compounds capable of binding to an insulin-like growth factor 1 (IGF1) receptor or an insulin receptor, comprising:
(a) determining an ability of a test compound to fit into a three-dimensional structure formed by a tyrosine kinase domain of the IGF1 receptor;
(b) determining an ability of the test compound to bind the insulin receptor; and
(c) selecting a test compound predicted to fit a three-dimensional structure formed by the tyrosine kinase domain of the IGF1 receptor, said test compound not capable of binding to the insulin receptor.

13. A computer-assisted method for designing a compound capable of binding a tyrosine kinase domain of an insulin-like growth factor-1 (IGF1) receptor, comprising:
(a) determining an ability of a test compound to fit into a three-dimensional structure formed by the tyrosine kinase domain of the IGF1 receptor (IGF1RK);
(b) generating the test compound;
(c) contacting the test compound with the three-dimensional IGF1RK structure; and
(d) determining if the test compound binds IGF1RK.

14. The method of claim 13, wherein the three-dimensional structure is the tyrosine kinase domain of the IGF1 receptor described by the coordinates of APPENDIX A.

15. The method of claim 13, wherein the tyrosine kinase domain of the IGF1 receptor comprises amino acid residues 992-1292 of SEQ ID NO: 1

16. The method of claim 13, wherein the computer-assisted method is virtual ligand docking and screening techniques capable of designing and/or identifying a compound predicted to bind to a three-dimensional motif of the tyrosine kinase domain of the IGF1 receptor.

17. The method of claim 16, wherein the three-dimensional motif of the tyrosine kinase domain of the IGF1 receptor is selected from the group consisting of an ATP-binding pocket, a peptide substrate binding groove, a hinge region on the backside of the kinase domain, and an alpha helix C.

18. The method of claim 13, wherein binding of the test compound to the IGF1RK is predicted to modulate an IGF1 receptor activity.

19. The method of claim 18, wherein binding of the test compound to the IGF1RK is predicted to reduce or inhibit an IGF1 receptor activity.

20. The method of claim 18, wherein binding of the test compound to the IGF1RK is predicted to enhance or prolong an IGF1 receptor activity.

21. The method of claim 18, wherein the IGF1 receptor activity is tyrosine kinase activity.

22. A computer-assisted method for designing a molecule capable of modulating an activity of an insulin-like growth factor-1 (IGF1) receptor, comprising:
(a) determining an ability of a test molecule to fit into a three-dimensional structure formed by a tyrosine kinase domain of the IGF1 receptor;
(b) selecting the test molecule predicted to bind the tyrosine kinase domain of the IGF1 receptor;
(c) generating the test molecule;
(d) contacting the test molecule with the three-dimensional IGF1RK structure; and
(e) determining if the test molecule binds IGF1RK, wherein a test molecule capable of binding to the IGF1RK and modulating an activity of the IGF1RK is a modulator of the IGF1 receptor.

23. The method of claim 22, wherein the three-dimensional structure is the tyrosine kinase domain of the IGF1 receptor having coordinates of APPENDIX A.

24. The method of claim 22, wherein the tyrosine kinase domain of the IGF1 receptor comprises amino acid residues 992-1292 of SEQ ID NO: 1

25. The method of claim 22, wherein the computer-assisted method is virtual ligand docking and screening techniques capable designing and/or identifying a compound predicted to bind to a three-dimensional motif of the tyrosine kinase domain of the IGF1 receptor.

26. The method of claim 25, wherein the three-dimensional motif of the tyrosine kinase domain of the IGF1 receptor is selected from the group consisting of an ATP-binding pocket, a peptide substrate binding groove, a hinge region on the backside of the kinase domain, and an alpha helix C.

27. The method of claim 22, wherein the modulator is capable of reducing or inhibiting IGF1RK activity.

28. The method of claim 22, wherein the modulator is capable of increasing or prolonging IGF1RK activity.

29. The method of claim 22, wherein the test molecule is a non-peptide-based molecule or a peptide-based molecule.

30. The method of claim 1, wherein the test compound is a non-peptide-based molecule or a peptide-based molecule.

31. The method of claim 12, wherein the test compound is a non-peptide-based molecule or a peptide-based molecule.

32. The method of claim 13, wherein the test compound is a non-peptide-based molecule or a peptide-based molecule.
